(19) United States
(12) Patent Application Publication
(10) Pub. No.: US 2012/0029016 A1
(43) Pub. Date: Feb. 2, 2012

(54) Indoles Useful in the Treatment of Inflammation

(75) Inventor: Peter Nilsson, Uppsala (SE)

(73) Assignee: BIOLIPOX AB, Uppsala (SW)

(21) Appl. No.: 13/142,825

(22) PCT Filed: Dec. 30, 2009
(86) PCT No.: PCT/GB2009/002977

§ 371 Date: Oct. 20, 2011

Related U.S. Application Data
(60) Provisional application No. 61/193,857, filed on Dec. 30, 2008.

(51) Int. Cl. A61K 31/404; C07D 401/12; A61K 31/4439; C07D 215/14; A61K 31/475; A61P 11/00; A61P 29/00; A61P 37/08; A61P 11/06; A61P 11/02; A61P 11/04; A61P 27/02; A61P 17/00; A61P 7/00; A61P 9/00; A61P 1/00; A61P 13/00; A61P 25/00; A61P 5/00; A61P 15/00; A61P 17/02; A61P 31/04; A61P 31/10; A61P 31/12; A61P 7/06; A61P 35/00; A61P 19/02; C07D 209/42

(52) U.S. Cl. 514/314; 548/491; 514/419; 546/278.1; 514/339; 546/174

(57) ABSTRACT

There is provided compounds of formula I, wherein ring R^{1a} to R^{1c}, R^{2a} to R^{2e}, and X have meanings given in the description, and pharmaceutically-acceptable salts thereof, which compounds are useful in the treatment of diseases in which inhibition of leukotriene C₄ synthase is desired and/or required, and particularly in the treatment of a respiratory disorder and/or inflammation.

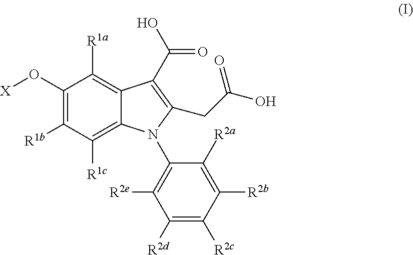

### FIELD OF THE INVENTION

This invention relates to novel pharmaceutically-useful compounds, which compounds are useful as inhibitors of the production of leukotrienes, such as leukotriene C₄. The compounds are of potential utility in the treatment of respiratory and/or inflammatory diseases. The invention also relates to the use of such compounds as medicaments, to pharmaceutical compositions containing them, and to synthetic routes for their production.

### BACKGROUND OF THE INVENTION

Arachidonic acid is a fatty acid that is essential in the body and is stored in cell membranes. It may be converted, e.g. in the event of inflammation, into mediators, some of which are known to have beneficial properties and others that are harmful. Such mediators include leukotrienes (formed by the action of 5-lipoxygenase (5-LO), which act by catalysing the insertion of molecular oxygen into carbon position 5) and prostaglandins (which are formed by the action of cyclooxygenases (COXs)). Huge efforts have been devoted towards the development of drugs that inhibit the action of these metabolites as well as the biological processes that form them.

Of the leukotrienes, leukotriene (LT) B₄ is known to be a strong proinflammatory mediator, while the cysteinyl-containing leukotrienes C₄, D₄ and E₄ (CysLTs) are mainly very potent bronchoconstrictors and have thus been implicated in the pathobiology of asthma. It has also been suggested that the CysLTs play a role in inflammatory mechanisms. The biological activities of the CysLTs are mediated through two receptors designated CysLT₁ and CysLT₂, but the existence of additional CysLT receptors has also been proposed. Leukotriene receptor antagonists (LTRAs) have been developed for the treatment of asthma, but they are often highly selective for CysLT₁. It may be hypothesised that better control of asthma, and possibly also chronic obstructive pulmonary disease (COPD), may be attained if the activity of both of the CysLT receptors could be reduced. This may be achieved by developing unselective LTRAs, but also by inhibiting the activity of proteins, e.g. enzymes, involved in the synthesis of the CysLTs; 5-LO, 5-lipoxygenase-activating protein (FLAP), and leukotriene C₄ synthase may be mentioned. However, a 5-LO or a FLAP inhibitor would also decrease the formation of LTB₄. For a review on leukotrienes in asthma, see H.-E Claesson and S.-E. Dahlén, J. Internal Med., 245, 205 (1999).

There are many diseases/disorders that are inflammatory in their nature or have an inflammatory component. One of the major problems associated with existing treatments of inflammatory conditions is a lack of efficacy and/or the prevalence of side effects (real or perceived).

Asthma is a chronic inflammatory disease affecting 6% to 8% of the adult population of the industrialised world. In children, the incidence is even higher, being close to 10% in most countries. Asthma is the most common cause of hospitalisation for children under the age of fifteen.

Treatment regimens for asthma are based on the severity of the condition. Mild cases are either untreated or are only treated with inhaled β-agonists. Patients with more severe asthma are typically treated with anti-inflammatory compounds on a regular basis.

There is a considerable under-treatment of asthma, which is due at least in part to perceived risks with existing maintenance therapy (mainly inhaled corticosteroids). These include risks of growth retardation in children and loss of bone mineral density, resulting in unnecessary morbidity and mortality. As an alternative to steroids, LTRAs have been developed. These drugs may be given orally, but are considerably less efficacious than inhaled steroids and usually do not control airway inflammation satisfactorily.

This combination of factors has led to at least 50% of all asthma patients being inadequately treated.

A similar pattern of under-treatment exists in relation to allergic disorders, where drugs are available to treat a number of common conditions but are underused in view of apparent side effects. Rhinitis, conjunctivitis and dermatitis may have an allergic component, but may also arise in the absence of underlying allergy. Indeed, non-allergic conditions of this class are in many cases more difficult to treat.

Chronic obstructive pulmonary disease (COPD) is a common disease affecting 6% to 8% of the world population. The disease is potentially lethal, and the morbidity and mortality from the condition is considerable. At present, there is no known pharmacological treatment capable of changing the course of COPD.

Other inflammatory disorders which may be mentioned include:

(a) pulmonary fibrosis (this is less common than COPD, but is a serious disorder with a very bad prognosis. No curative treatment exists);
(b) inflammatory bowel disease (a group of disorders with a high morbidity rate. Today only symptomatic treatment of such disorders is available); and
(c) rheumatoid arthritis and osteoarthritis (common disabling inflammatory disorders of the joints. There are currently no curative, and only moderately effective symptomatic, treatments available for the management of such conditions).

Inflammation is also a common cause of pain. Inflammatory pain may arise for numerous reasons, such as infection, surgery or other trauma. Moreover, several malignancies are known to have inflammatory components adding to the symptomatology of the patients.

Thus, new and/or alternative treatments for respiratory and/or inflammatory disorders would be of benefit to all of the above-mentioned patient groups. In particular, there is a real and substantial unmet clinical need for an effective anti-inflammatory drug capable of treating inflammatory disorders, in particular asthma and COPD, with no real or perceived side effects.

The listing or discussion of an apparently prior-published document in this specification should not necessarily be taken as an acknowledgement that the document is part of the state of the art or is common general knowledge.

International patent application WO 2006/077366 discloses indoles as mPGES-1 inhibitors, which are therefore useful in the treatment of inflammation. International patent application WO 2008/097930 discloses various indoles for use as inhibitors of 5-lipoxygenase activating protein. However, there is no specific disclosure in either of these documents of indole-3-carboxylates, substituted at the 2-position with a carboxymethyl group, substituted at the 5-position with group linked with an oxygen linker group, and directly substituted at the 1(N)-position with a phenyl group, which is itself substituted with certain substituents.

Various other indoles (or variants thereof) have also been disclosed in international patent applications WO 2005/005415, WO 2005/123673, WO 2005/123674, WO 2005/123675, WO 2006/077364, WO 2006/077367, WO 2006/077365, WO 2006/077412 and WO 2006/077401 for use as mPGES-1 inhibitors (which compounds may therefore be of use in the treatment of inflammation). However, there is no disclosure in any of those documents of 3-carboxy indoles.

### SUMMARY OF THE INVENTION

There is provided a compound of formula I,

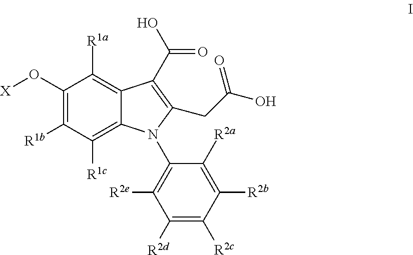

wherein:
R^{1a}, R^{1b} and R^{1c} independently represent hydrogen, halo, —CN or C₁₋₃ alkyl optionally substituted by one or more fluoro atoms;
any one of R^{2a}, R^{2b}, R^{2c}, R^{2d} and R^{2e} represents -L-Y, and the others independently represent hydrogen, halo, —CN or C₁₋₃ alkyl optionally substituted by one or more fluoro atoms;
X represents:

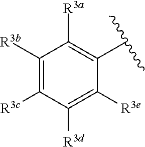

R^{3a}, R^{3b}, R^{3c}, R^{3d} and R^{3e} independently represent hydrogen, halo, —CN, R^{a}, —N(R^{b})R^{c}, —OR^{d} or —S(O)ₙR^{e};
L represents a direct bond, —N(R^{y})—, —O— or —S(O)ₘ—;
Y represents:

### (i) G¹;

### (ii) —CH₂-G²;

(iii) cyclopropyl optionally substituted by one or more substituents independently selected from G³;
(iv) ethyl, 1-propyl (i.e. n-propyl) or 2-propyl (i.e. isopropyl), all of which are substituted by one or more substituents independently selected from G⁴; or
(v) C₄₋₁₅ alkyl optionally substituted by one or more substituents independently selected from G⁵;
R^{a} and R^{e} independently represent C₁₋₆ alkyl optionally substituted by one or more fluoro atoms;
R^{b}, R^{c}, R^{d} and R^{y} independently represent hydrogen or C₁₋₆ alkyl optionally substituted by one or more fluoro atoms;
G¹ and G² independently represent an aryl group or a heteroaryl group, both of which groups are optionally substituted by one or more substituents independently selected from G⁶;
G³, G⁴ and G⁵ independently represent fluoro, —OR^{f}, aryl or heteroaryl, which latter two groups are optionally substituted by one or more substituents independently selected from G⁷;
G⁶ and G⁷ independently represent, on each occasion when used herein, halo, —CN, R^{g}, —N(R^{h})Rⁱ, —OR^{j} or —S(O)ₚR^{k};
R^{f}, R^{h}, Rⁱ and R^{j} independently represent, on each occasion when used herein, hydrogen or C₁₋₆ alkyl optionally substituted by one or more fluoro atoms;
R^{g} and R^{k} independently represent C₁₋₆ alkyl optionally substituted by one or more fluoro atoms;
n, m and p independently represent 0, 1 or 2,
or a pharmaceutically-acceptable salt thereof,
which compounds and salts are referred to hereinafter as “the compounds of the invention”.

Pharmaceutically-acceptable salts include acid addition salts and base addition salts. Such salts may be formed by conventional means, for example by reaction of a free acid or a free base form of a compound of formula I with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of the invention in the form of a salt with another counter-ion, for example using a suitable ion exchange resin.

Compounds of the invention may contain double bonds and may thus exist as E (entgegen) and Z (zusammen) geometric isomers about each individual double bond. All such isomers and mixtures thereof are included within the scope of the invention.

Compounds of the invention may also exhibit tautomerism. All tautomeric forms and mixtures thereof are included within the scope of the invention.

Compounds of the invention may also contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise specified, C_{1-q} alkyl (where q is the upper limit of the range), defined herein may be straight-chain or, when there is a sufficient number (i.e. a minimum of three) of carbon atoms, be branched-chain, and/or cyclic (so forming, in the case of alkyl, a C_{3-q} cycloalkyl group; which may include cycloalkyl (e.g. C_{3-q1} cycloalkyl, wherein q1 is the upper limit of the range) substituted by alkyl (e.g. C_{1-q1} alkyl), provided that the total number of carbon atoms does not exceed the upper limit, i.e. the sum of the respective q1 groups does not exceed the value of q). Further, when there is a sufficient number (i.e. a minimum of four) of carbon atoms, such groups may also be part cyclic (i.e. in which the cycloalkyl moiety is a sub-moiety of an alkyl group, for example it may include a C_{1-q1} alkyl group substituted by a C_{3-q1} cycloalkyl group or vice versa). Further, unless otherwise specified, such alkyl groups may also be saturated or, when there is a sufficient number (i.e. a minimum of two) of carbon atoms and unless otherwise specified, be unsaturated (forming, for example, a C_{2-q} alkenyl or a C_{2-q} alkynyl group).

In the instance where a ‘cycloalkyl’ group (e.g. C_{3-q} cycloalkyl) is specifically mentioned, such groups may be monocyclic or bicyclic non-aromatic alkyl groups, which may be bridged (so forming, for example, fused ring systems). In the context of the invention, a ‘cycloalkyl’ group also includes a part-cyclic group as defined above. As specified above, the cycloalkyl group may be substituted by one or more alkyl moieties (provided that the total number of carbon atoms does not exceed the upper limit) as well as other substituents defined herein. Cycloalkyl groups may also include spiro-cyclic groups. Cycloalkyl groups may be saturated or unsaturated, e.g. containing one or more double bond (forming for example a C_{3-q} cycloalkenyl). Optional substituents may be attached at any point on the cycloalkyl group. Cycloalkyl groups that may be mentioned include C₃₋₁₂ cycloalkyl groups, for instance a 3- to 7-membered monocyclic cycloalkyl group or a C₈₋₁₁ bicyclic cycloalkyl group (both of which are optionally substituted with one or more C₁₋₆ (e.g. C₁₋₂) alkyl (e.g. methyl) groups and one or more substituents as defined herein). The term ‘acyclic’ alkyl group when used herein refers to an alkyl group that is not cyclic, but may be branched-chain or, is preferably, straight-chain.

For the avoidance of doubt, the term “bicyclic”, when employed in the context of cycloalkyl, refers to such groups in which the second ring is formed between two adjacent atoms of the first ring (i.e. systems of two rings share one bond formed with two adjacent carbon atoms). The term “bridged”, when employed in the context of cycloalkyl groups refers to cycloalkyl groups in which two non-adjacent atoms are linked by an alkylene chain (so forming, for example a norbornanyl group). The term “spiro-cyclic group” refers to a cycloalkyl group that is substituted with a further cycloalkyl group via a single carbon atom.

The term “halo”, when used herein, includes fluoro, chloro, bromo and iodo.

Aryl groups that may be mentioned include C₆₋₁₄ (such as C₆₋₁₃ (e.g. C₆₋₁₀)) aryl groups. Such groups may be monocyclic or bicyclic and have between 6 and 14 ring carbon atoms, in which at least one ring is aromatic. C₆₋₁₄ aryl groups include phenyl, naphthyl and the like, such as 1,2,3,4-tetrahydronaphthyl, indanyl, indenyl and fluorenyl. The point of attachment of aryl groups may be via any atom of the ring system. However, when aryl groups are bicyclic or tricyclic, they are preferably linked to the rest of the molecule via an aromatic ring.

Heteroaryl groups that may be mentioned include those which have between 5 and 14 (e.g. 10) members. Such groups may be monocyclic, bicyclic or tricyclic, provided that at least one of the rings is aromatic and wherein at least one (e.g. one to four) of the atoms in the ring system is other than carbon (i.e. a heteroatom). Heteroaryl groups that may be mentioned include acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothiadiazolyl (including 2,1,3-benzothiadiazolyl), benzothienyl, carbazolyl, chromanyl, cinnolinyl, furanyl, imidazolyl, imidazopyridyl (including imidazo[4,5-b]pyridyl, imidazo[5,4-b]pyridyl and imidazo[1,2-a]pyridyl), indazolyl, indolinyl, indolyl, isobenzofuranyl, isochromanyl, isoindolinyl, isoindolyl, isoquinolinyl, isothiazolyl, isothiochromanyl, isoxazolyl, naphthyridinyl (including 1,6-naphthyridinyl or, preferably, 1,5-naphthyridinyl and 1,8-naphthyridinyl), oxadiazolyl (including 1,3,4-oxadiazolyl), oxazolyl, phenazinyl, phenothiazinyl, phthalazinyl, pteridinyl, purinyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,3,4-thiadiazolyl), thiazolyl, oxazolopyridyl (including oxazolo[4,5-b]pyridyl, oxazolo[5,4-b]pyridyl and, in particular, oxazolo[4,5-c]pyridyl and oxazolo[5,4-c]pyridyl), thiazolopyridyl (including thiazolo[4,5-b]pyridyl, thiazolo[5,4-b]pyridyl and, in particular, thiazolo[4,5-c]pyridyl and thiazolo[5,4-c]pyridyl), thiochromanyl, thienyl, triazolyl (including 1,2,3-triazolyl and 1,2,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. However, when heteroaryl groups are polycyclic, they are preferably linked to the rest of the molecule via an aromatic ring. Heteroaryl groups may also be in the N- or S-oxidised form.

Heteroatoms that may be mentioned include phosphorus, silicon, boron, tellurium, selenium and, preferably, oxygen, nitrogen and sulphur.

For the avoidance of doubt, when a phrase such as “R^{3a} to R^{3e}” is employed herein, this will be understood by the skilled person to mean R^{3a}, R^{3b}, R^{3c}, R^{3d} and R^{3e} inclusively.

For the avoidance of doubt, in cases in which the identity of two or more substituents in a compound of formula I may be the same, the actual identities of the respective substituents are not in any way interdependent. For example, in the situation in which two or more R^{g} groups are present and represent C₁₋₆ alkyl, the respective alkyl groups may be the same or different. Hence, where there is, for example, more than one C₁₋₆ alkyl group present in the compound of formula I, then such groups are not in any way interdependent, i.e. they may be the same or different.

Compounds of the invention that may be mentioned include those in which:

Y represents: (i) G¹; (ii) —CH₂-G²; (iii) C₃₋₁₅ cycloalkyl optionally substituted by one or more substituents independently selected from G³ and/or G⁵ (as appropriate); (iv) acyclic C₄₋₁₅ alkyl optionally substituted by one or more substituents independently selected from G⁵;
when Y represents optionally substituted C₃₋₁₅ cycloalkyl, then it may represent optionally substituted C₃₋₈ cycloalkyl in which the cycloalkyl group is cyclic (e.g. cyclopentyl, cyclohexyl, norbornanyl, all of which may be substituted by one or more (e.g. one or two) C₁₋₃ (e.g. C₁₋₂) alkyl (e.g. methyl) group, provided that the upper limit of carbon atoms is not exceeded, so forming for example, a 4,4-dimethylcyclohexyl group, and all of which may be further optionally substituted as defined herein, i.e. by one or more G³ and/or G⁵ substituents), or optionally substituted C₄₋₈ cycloalkyl in which the group is part cyclic (e.g. C₁₋₆ alkyl substituted by C₃₋₇ cycloalkyl (in which the latter group is cyclic) (e.g. cyclohexylmethyl), which is optionally substituted as defined herein, i.e. by one or more G³ and/or G⁵ substituents);
when Y represents optionally substituted acyclic C₄₋₁₅ alkyl, then it preferably represents acyclic C₄₋₁₀ (e.g. C₆₋₈) alkyl.

Preferred compounds of the invention include those in which:

R^{1a}, R^{1b} or R^{1c} independently represent hydrogen, halo (e.g. fluoro or chloro) or methyl optionally substituted by one or more fluoro atoms (so forming, for example, a trifluoromethyl group);
any one of R^{2b}, R^{2c} and R^{2d} (preferably R^{2c}) represents -L-Y, and the others independently represent hydrogen, halo (e.g. fluoro or chloro), —CN or C₁₋₃ alkyl optionally substituted by one to three fluoro atoms (so forming, for example, a trifluoromethyl group);
R^{2a} and R^{2e} independently represent hydrogen, halo (e.g. fluoro or chloro) or methyl optionally substituted by one or more fluoro atoms (so forming, for example, a trifluoromethyl group);
Y represents G¹, —CH₂-G² or C₄₋₁₅ alkyl optionally substituted by one or more substituents independently selected from G⁵;
R^{3a}, R^{3b}, R^{3c}, R^{3d} and R^{3e} independently represent hydrogen, halo (e.g. fluoro or chloro), —CN, R^{a} or —OR^{d};
one of R^{3a}, R^{3b}, R^{3c}, R^{3d} and R^{3e} (e.g. one of R^{3b}, R^{3c} and R^{3d}) represents a substituent other than hydrogen (e.g. —N(R^{b})R^{c} or, preferably, —OR^{d}), and the others independently represent fluoro, chloro, methyl, trifluoromethyl or, preferably, hydrogen;
R^{a} and R^{d} independently represent C₁₋₄ (e.g. C₁₋₃) alkyl (e.g. isopropyl or methyl) optionally substituted by one to three fluoro atoms (so forming, for example, a trifluoromethyl group);
R^{b} and R^{c} independently represent hydrogen or C₁₋₄ (e.g. C₁₋₃) alkyl (e.g. isopropyl or methyl) optionally substituted by one to three fluoro atoms (so forming, for example, a trifluoromethyl group);
at least one of R^{b} and R^{c} is other than hydrogen;
G¹ and G² independently represent: aryl (e.g. phenyl); naphthyl (e.g. in which one of the rings is saturated or partially saturated, e.g. 5,6,7,8-tetrahydronaphthyl); a 5- or 6-membered monocyclic heteroaryl ring (e.g. containing two or, preferably, one heteroatom preferably selected from oxygen and, particularly, nitrogen; so forming, for example, a pyridyl group, e.g. a 2- or 3-pyridyl group); or a 9- or 10-membered bicyclic heteroaryl group (e.g. containing two or, preferably, one heteroatom preferably selected from sulfur, particularly, oxygen and, more particularly, nitrogen; so forming, for example an quinolinyl, e.g. 2-quinolinyl group), all of which aryl and heteroaryl groups are optionally substituted by one or more substituents selected from G⁶;
G⁵ represents aryl or heteroaryl, which latter two groups may be substituted by one or more G⁷ substituents;
G⁶ and G⁷ independently represent halo (e.g. bromo, fluoro or chloro), —CN, R^{g}, —OR^{j} or —S(O)ₙR^{k};
R^{j} represents hydrogen or, preferably, C₁₋₃ alkyl optionally substituted by one to three fluoro atoms;
R^{g} and R^{k} independently represent C₁₋₃ alkyl optionally substituted by one to three fluoro atoms.

Preferred aryl and heteroaryl groups that G¹, G² or G⁵ may represent include optionally substituted (i.e. by G⁶ or G⁷) phenyl, naphthyl, pyrrolyl, furanyl, thienyl (e.g. 2-thienyl or 3-thienyl), imidazolyl (e.g. 2-imidazolyl or 4-imidazolyl), oxazolyl, isoxazolyl, thiazolyl, pyrazolyl, pyridyl (e.g. 2-pyridyl, 3-pyridyl or 4-pyridyl), indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzoxazolyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, and/or benzodioxanyl, group. Preferred groups include isoquinolinyl and, preferably, pyridyl (e.g. 2- or 3-pyridyl), quinolinyl (e.g. 2-quinolinyl), naphthyl (e.g. 1-napthyl, such as 5,6,7,8-tetrahydro-1-naphthyl) and phenyl.

Particularly preferred compounds of the invention include those in which:

any one of R^{1a}, R^{1b} and R^{1c} represents halo (e.g. fluoro or chloro), methyl or trifluoromethyl, and the others represents hydrogen or, more preferably, R^{1a}, R^{1b} and R^{1c} independently represent hydrogen (e.g. all of R^{1a}, R^{1b} and R^{1c} represent hydrogen);
R^{2a} and R^{2e} independently represent hydrogen;
R^{y} represents hydrogen or C₁₋₂ alkyl (e.g. methyl or ethyl);
one of R^{2b} and R^{2d} represents fluoro, chloro, methyl or trifluoromethyl, and the other represents hydrogen or, more preferably, R^{2b} and R^{2d} independently represent hydrogen (e.g. both represent hydrogen);
R² represent -L-Y;
L represents a direct bond or —N(R^{y})— or, more preferably, —O—.

Preferred substituents that G⁶ may represent include —NH₂, —N(H)(CH₃), —N(CH₃)₂, —OH and, preferably, halo (e.g. fluoro and/or chloro), C₁₋₃ (e.g. C₁₋₂) alkoxy (e.g. methoxy), —S—C₁₋₃ alkyl (e.g. —S—CH₃; optionally substituted by one or more fluoro atoms, so forming for example, and C₁₋₃ (e.g. C₁₋₂) alkyl (e.g. methyl), which latter group may itself be optionally substituted by one or more fluoro atom (so forming, for example, a trifluoromethyl group). Preferred substituents that G³, G⁴ and G⁵ may represent include fluoro, aryl (e.g. phenyl) and heteroaryl (e.g. a 5- or 6-membered monocyclic heteroaryl group in which the heteroatom(s) is/are preferably selected from oxygen and, particularly, nitrogen; so forming for example a pyridyl group, e.g. 2-pyridyl).

Particularly preferred compounds of the invention include those in which:

R^{3c} represents —OR^{d};
R^{3a}, R^{3b}, R^{3d} and R^{3e} independently represent hydrogen;
L represents —N(R^{y})— or —O—;
R^{y} represents hydrogen or C₁₋₂ alkyl (e.g. ethyl);
Y represents: (i) G¹; (ii) —CH₂-G²; (iii) C₄₋₁₅ (e.g. C₄₋₁₀, such as C₄₋₈) alkyl (e.g. C₄₋₈ part cyclic alkyl, such as methyl substituted by C₅₋₆ cycloalkyl (e.g. cyclohexylmethyl); acyclic C₄₋₁₅ (e.g. C₆₋₁₀, such as C₆₋₈) alkyl, such as octyl; or C₅₋₈ cycloalkyl, such as cyclopentyl, cyclohexyl, 4,4-dimethylcyclohexyl or norbornanyl), optionally substituted by one or more substituents selected from G⁵;
G¹ and G² independently represent phenyl, pyridyl (e.g. 2- or 3-pyridyl), quinolinyl (e.g. 2-quinolinyl) or naphthyl (e.g. 5,6,7,8-tetrahydronaphthyl), all of which are optionally substituted by one or more G⁶ groups;
G⁴ represents fluoro;
G⁵ represents —OR^{f}, preferably, optionally substituted (as defined herein) aryl or heteroaryl, or, more preferably, G⁵ represents fluoro;
G⁶ and G⁷ independently represent halo (e.g. bromo, fluoro or, preferably, chloro), —OR^{j}, R^{g} or —S(O)ₚR^{k} (e.g. —S—R^{k});
R^{j}, R^{g} and R^{k} independently represent C₁₋₃ (e.g. C₁₋₂) alkyl optionally substituted by one or more fluoro atoms (so forming e.g. a —CF₃ group).

Particularly preferred G¹ and G² groups include unsubstituted phenyl, methylphenyl (e.g. 4-methylphenyl), chlorophenyl (e.g. 2-, 3- or 4-chlorophenyl), dichlorophenyl (e.g. 3,4-dichlorophenyl), methoxyphenyl (e.g. 4-methoxyphenyl), trifluoromethylphenyl (e.g. 2-trifluoromethylphenyl), (trifluoromethylthio)phenyl (e.g. (4-trifluoromethylthio)phenyl), unsubstituted pyridyl (e.g. 2-pyridyl), chloropyridyl (e.g. 6-chloro-3-pyridyl or 2-chloro-5-pyridyl), unsubstituted quinolinyl (e.g. 2-quinolinyl) and unsubstituted naphthyl (e.g. 1-naphthyl, such as 5,6,7,8-tetrahydro-1-naphthyl). Particularly preferred C₄₋₁₅ alkyl groups include C₅₋₈ cycloalkyl groups such as cyclopentyl, cyclohexyl and norbornanyl (optionally substituted for example by C₁₋₂ alkyl groups (e.g. methyl) so forming for example a 4,4-dimethylcyclohexyl group), and acyclic C₁₋₈ alkyl (e.g. methyl or octyl; optionally substituted, for example by C₁₋₆ alkyl, e.g. C₅₋₆ cycloalkyl, so forming for example a cyclohexylmethyl group).

Particularly preferred compounds of the invention include those of the examples described hereinafter.

Compounds of the invention may be made in accordance with techniques that are well known to those skilled in the art, for example as described hereinafter.

According to a further aspect of the invention there is provided a process for the preparation of a compound of formula I which process comprises:

(i) reaction of a compound of formula II,

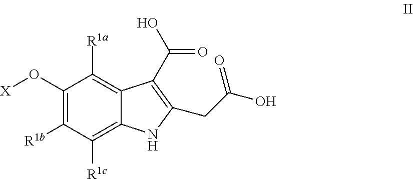

wherein R^{1a}, R^{1c} and X are as hereinbefore defined, with a compound of formula III,

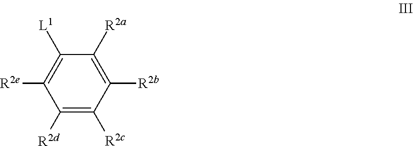

wherein L¹ represents a suitable leaving group such as chloro, bromo, iodo, a sulfonate group (e.g. —OS(O)₂CF₃, —OS(O)₂CH₃, —OS(O)₂PhMe or a nonaflate) or —B(OH)₂, or a protected derivative thereof (such as an alkyl protected derivative, so forming, for example a 4,4,5,5-tetramethyl-1,3,2-dioxaborolan-2-yl group), and R^{2a}, R^{2b}, R^{2c}, R^{2d} and R^{2e} are as hereinbefore defined, for example optionally in the presence of an appropriate metal catalyst, e.g. a metal (or a salt or complex thereof) such as Cu, Cu(OAc)₂, CuI (or CuI/diamine complex), copper tris(triphenyl-phosphine)bromide, Pd(OAc)₂, Pd₂(dba)₃ or NiCl₂ and an optional additive such as Ph₃P, 2,2′-bis(diphenylphosphino)-1,1′-binaphthyl, xantphos, NaI or an appropriate crown ether such as 18-crown-6-benzene, in the presence of an appropriate base such as NaH, Et₃N, pyridine, N,N′-dimethylethylenediamine, Na₂CO₃, K₂CO₃, K₃PO₄, Cs₂CO₃, t-BuONa or t-BuOK (or a mixture thereof, optionally in the presence of 4 Å molecular sieves), in a suitable solvent (e.g. dichloromethane, dioxane, toluene, ethanol, isopropanol, dimethylformamide, ethylene glycol, ethylene glycol dimethyl ether, water, dimethylsulfoxide, acetonitrile, dimethylacetamide, N-methylpyrrolidinone, tetrahydrofuran or a mixture thereof) or in the absence of an additional solvent when the compound of formula III may itself act as a solvent. This reaction may be carried out at room temperature or above (e.g. at a high temperature, such as the reflux temperature of the solvent system that is employed) or using microwave irradiation;
(ii) reaction of a compound of formula IV,

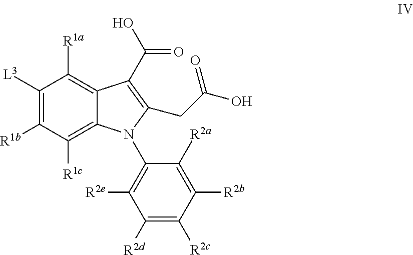

wherein L³ represents a suitable leaving group, such as one hereinbefore defined in respect of L² (for example —B(OH)₂), or a protected derivative thereof), and R^{1a} to R^{1c} and R^{2a} to R^{2e} are as hereinbefore defined with a compound of formula V,

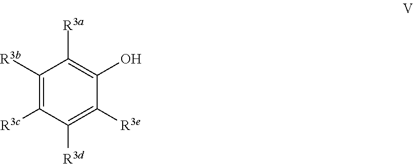

wherein R^{3a} to R^{3e} are as hereinbefore defined, for example under reaction conditions such as those hereinbefore described in respect of process step (ii) above, e.g. in the presence of an appropriate metal catalyst (or a salt or complex thereof) such as Cu(OAc)₂, an appropriate base such as Et₃N and/or pyridine and a suitable solvent (e.g. dichloromethane and/or dimethylformamide);
(iii) reaction of a compound of formula VI,

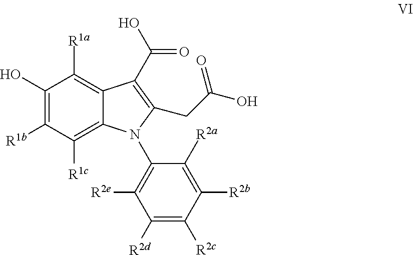

wherein R^{1a} to R^{1c} and R^{2a} to R^{2e} are as hereinbefore defined with a compound of formula VII,

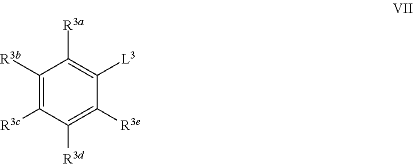

wherein L³ is as hereinbefore defined (for example —B(OH)₂, chloro, bromo or iodo), and R^{3a} to R^{3e} are as hereinbefore defined, under conditions such as those hereinbefore described in respect of process step (ii) above;
(iv) reaction of a protected derivative of a compound of formula VIII,

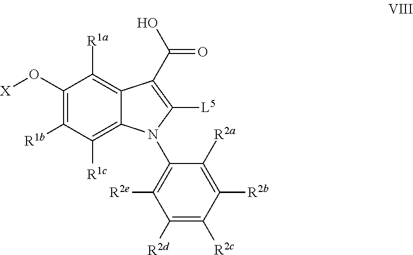

wherein L⁵ represents an appropriate alkali metal group (e.g. sodium, potassium or, especially, lithium), a —Mg-halide, a zinc-based group or a suitable leaving group such as halo or —B(OH)₂, the —COOH is protected, for example, as the carboxylic acid ester (e.g. —COO—C₁₋₆ alkyl), and R^{1a} to R^{1c}, R^{2a} to R^{2e} and X are as hereinbefore defined, with a protected derivative of a compound of formula IX,

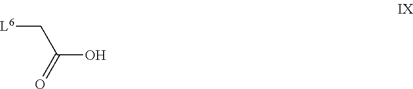

wherein the —COOH is protected, for example, as the carboxylic acid ester (e.g. —COO—C₁₋₆ alkyl), and L⁶ represents a suitable leaving group, such as halo (especially chloro or bromo). The reaction may be performed under suitable reaction conditions, for example by:
(i) forming the corresponding Grignard reagent under standard conditions known to those skilled in the art (e.g. employing magnesium or a suitable reagent such as a mixture of C₁₋₆ alkyl-Mg-halide and ZnCl₂ or LiCl) followed by reaction with a compound of formula XI, optionally in the presence of a catalyst (e.g. FeCl₃) under conditions known to those skilled in the art; or
(ii) forming the corresponding lithiated compound under halogen-lithium exchange reaction conditions known to those skilled in the art (e.g. employing n-BuLi or t-BuLi in the presence of a suitable solvent (e.g. a polar aprotic solvent such as THF)), followed by reaction with a compound of formula XI;
   followed by (if necessary) deprotection under standard conditions. The skilled person will also appreciate that L⁵ and L⁶ (when they both represent leaving groups) will be mutually compatible;
   (v) reaction of a protected derivative of a compound of formula X,

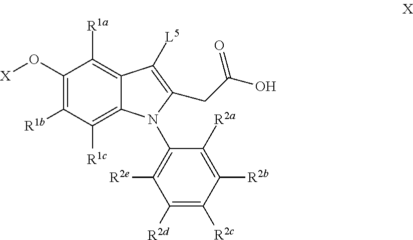

wherein the —COOH may be protected, for example, as the carboxylic acid ester (e.g. —COO—C₁₋₆ alkyl), and L⁵, R^{1a} to R^{1c}, R^{2a} to R^{2e} and X are as hereinbefore defined, with a protected derivative of a compound of formula XI,

L⁶-C(O)OH XI

wherein the —COOH is protected, for example, as the carboxylic acid ester (e.g. —COO—C₁₋₆ alkyl), L⁶ is as hereinbefore defined, for example under similar reaction conditions to those described hereinbefore in respect of process (iv) above;
(vi) reaction of a compound of formula X in which L⁵ represents either:
(I) an alkali metal (for example as defined in respect of process step (iv) above); or

### (II) —Mg-halide,

with carbon dioxide, followed by acidification under standard conditions known to those skilled in the art, for example, in the presence of aqueous hydrochloric acid;
(vii) for compounds of formula I, and protected derivatives thereof (e.g. those in which the —COOH group is protected so forming an ester e.g. a —COO—C₁₋₆ alkyl group), reaction of a corresponding compound of formula X as hereinbefore defined in which L⁵ is a suitable leaving group known to those skilled in the art (such as a sulfonate group (e.g. a triflate) or, preferably, a halo (e.g. bromo or iodo) group) with CO (or a reagent that is a suitable source of CO (e.g. Mo(CO)₆ or CO₂(CO)₈), in the presence of a compound of formula XII,

R⁴—OH XII

wherein R⁴ represents hydrogen or a suitable protecting group (ultimately for a carboxylic acid ester group), such as C₁₋₆ alkyl, and an appropriate catalyst system (e.g. a palladium catalyst such as one described hereinbefore in respect of process step (i)) under conditions known to those skilled in the art;
(viii) for compounds of formula I in which L represents —O—, —N(R^{y})— or —S—, reaction of a compound corresponding to a compound of formula I, but in which Y represents hydrogen, with a compound of formula XIII,

L⁷-Y XIII

wherein L⁷ represents a suitable leaving group, such as chloro, bromo, iodo, a sulfonate group (e.g. —OS(O)₂CF₃, —OS(O)₂CH₃, —OS(O)₂PhMe or a nonaflate), —B(OH)₂ (or a protected derivative thereof, e.g. an alkyl protected derivative e.g. —B(ORⁿ)₂, in which Rⁿ is a C₁₋₆ alkyl group or the protected derivative may form e.g. a 4,4,5,5-tetramethyl-1,3,2-dioxaborolan-2-yl group), —Sn(Rⁿ)₃ (in which Rⁿ is a C₁₋₆ alkyl group such as ethyl or methyl), —BF₃K (or the like) or diazonium salts. Alternatively, L⁷ may represent —OH, and the reactivity of the —OH group may be enhanced by reaction under Mitsunobu reaction conditions, e.g. in the presence of triphenylphosphine and an azo compound, such as DEAD or the like), and Y is as hereinbefore defined, under reaction conditions known to those skilled in the art, for example:
(I) when Y represents —CH₂-G² or acyclic alkyl (i.e. ethyl, 1-propyl or 2-propyl, all of which are substituted as herein defined, or, acyclic C₄₋₁₅ alkyl optionally substituted as defined herein), the reaction may be performed at around room temperature or above (e.g. up to 40-180° C.), optionally in the presence of a suitable base (e.g. sodium hydride, sodium bicarbonate, potassium carbonate, pyrrolidinopyridine, pyridine, triethylamine, tributylamine, trimethylamine, dimethylaminopyridine, diisopropylamine, diisopropylethylamine, 1,8-diazabicyclo[5.4.0]undec-7-ene, sodium hydroxide, N-ethyldiisopropylamine, N-(methylpolystyrene)-4-(methylamino)pyridine, potassium bis(trimethylsilyl)amide, sodium bis(trimethylsilyl)amide, potassium tert-butoxide, lithium diisopropylamide, lithium 2,2,6,6-tetramethylpiperidine or mixtures thereof) and an appropriate solvent (e.g. tetrahydrofuran, pyridine, toluene, dichloromethane, chloroform, acetonitrile, dimethylformamide, trifluoromethylbenzene, dioxane or triethylamine); or
(II) when Y represents G¹ or cycloalkyl (directly linked to L; i.e. cyclopropyl or C₄₋₁₅ cycloalkyl, both of which are optionally substituted as defined herein), under reaction conditions such as those hereinbefore described in respect of process step (i) above;
   (ix) for compounds of formula I in which L represents —O—, —N(R^{y})— or —S—, reaction of a compound corresponding to a compound of formula I, but in which -L-Y represents a suitable leaving group, such as one hereinbefore defined by L⁷, with a compound of formula XIV,

L^{x1}-Y XIV

wherein L^{x1} represents —OH, —N(R^{y})H or —SH, and R^{y} and Y are as hereinbefore defined, for example under reaction conditions such as those hereinbefore described in respect of process step (i) above.

Compounds of formula VI (or protected derivatives thereof) in which R^{1a}, R^{1b} and R^{1c} all represent hydrogen, may be prepared by reaction of a compound of formula XV,

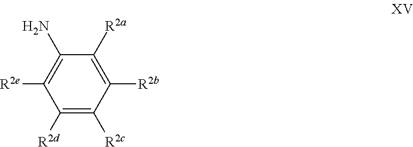

or protected derivatives thereof, wherein R^{2a} to R^{2e} are as hereinbefore defined, with a compound of formula XVI,

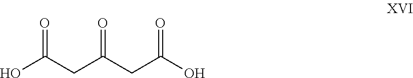

or protected derivatives thereof (e.g. in which one or both of the —COOH groups are protected), in the presence of a suitable catalyst, for example an sulfonic acid (e.g. p-toluenesulfonic acid), followed by subsequent reaction with 1,4-benzoquinone, performed at around room temperature or above (e.g. up to 40-180° C.), under conditions known to those skilled in the art.

Compounds of formula X in which L⁵ represents halo may be prepared by reaction of a compound corresponding to a compound of formula X, but in which L⁵ represents hydrogen, with a reagent or mixture of reagents known to be a source of halide atoms.

For example, for bromide atoms, N-bromosuccinimide, bromine or 1,2-dibromotetrachloroethane may be employed, for iodide atoms, iodine, diiodoethane, diiodotetrachloroethane or a mixture of NaI or KI and N-chlorosuccinimide may be employed, for chloride atoms, N-chlorosuccinimide may be employed and for fluoride atoms, 1-(chloromethyl)-4-fluoro-1,4-diazoniabicyclo[2.2.2]octane bis(tetra-fluoroborate), 1-fluoropyridinium triflate, xenon difluoride, CF₃OF or perchloryl fluoride may be employed. This reaction may be carried out in a suitable solvent (e.g. acetone, benzene or dioxane) under conditions known to the skilled person.

Compounds of formula X in which L⁵ represents a sulfonate group, may be prepared by reaction of a compound corresponding to a compound of formula X, but in which L⁵ represents —OH, with an appropriate reagent for the conversion of the hydroxyl group to the sulfonate group (e.g. tosyl chloride, mesyl chloride, triflic anhydride and the like) under conditions known to those skilled in the art.

Compounds of formulae II, III, IV, V, VI, VII, VIII, IX, X, XI, XII, XIII, XIV, XV and XVI are either commercially available, are known in the literature, or may be obtained either by analogy with the processes described herein, or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991 and/or to the procedures described in international patent application WO 2006/077366.

Indoles may also be prepared with reference to a standard heterocyclic chemistry textbook (e.g. “Heterocyclic Chemistry” by J. A. Joule, K. Mills and G. F. Smith, 3rd edition, published by Chapman & Hall or “Comprehensive Heterocyclic Chemistry II” by A. R. Katritzky, C. W. Rees and E. F. V. Scriven, Pergamon Press, 1996) and/or made according to the procedures described in international patent application WO 2006/077366.

The substituents in final compounds of the invention or relevant intermediates may be modified one or more times, after or during the processes described above by way of methods that are well known to those skilled in the art. Examples of such methods include substitutions, reductions, oxidations, alkylations, acylations, hydrolyses, esterifications, etherifications, halogenations or nitrations. The precursor groups can be changed to a different such group, or to the groups defined in formula I, at any time during the reaction sequence. For example, in cases where R³ or R⁴ do not initially represent hydrogen (so providing at least one ester functional group), the skilled person will appreciate that at any stage during the synthesis (e.g. the final step), the relevant R³- or R⁴-containing group may be hydrolysed to form a carboxylic acid functional group (i.e. a group in which R³ or R⁴ represents hydrogen). In this respect, the skilled person may also refer to “Comprehensive Organic Functional Group Transformations” by A. R. Katritzky, O. Meth-Cohn and C. W. Rees, Pergamon Press, 1995.

Other transformations that may be mentioned include: the conversion of a halo group (preferably iodo or bromo) to a —CN or 1-alkynyl group (e.g. by reaction with a compound which is a source of cyano anions (e.g. sodium, potassium, copper (I) or zinc cyanide) or with a 1-alkyne, as appropriate), which latter reaction may be performed in the presence of a suitable coupling catalyst (e.g. a palladium and/or a copper based catalyst) and a suitable base (e.g. a tri-(C₁₋₆ alkyl)amine such as triethylamine, tributylamine or ethyldiisopropylamine); the introduction of amino groups and hydroxy groups in accordance with standard conditions using reagents known to those skilled in the art; the conversion of an amino group to a halo, azido or a cyano group, for example via diazotisation (e.g. generated in situ by reaction with NaNO₂ and a strong acid, such as HCl or H₂SO₄, at low temperature such as at 0° C. or below, e.g. at about −5° C.) followed by reaction with the appropriate nucleophile e.g. a source of the relevant anions, for example by reaction in the presence of a halogen gas (e.g. bromine, iodine or chlorine), or a reagent that is a source of azido or cyanide anions, such as NaN₃ or NaCN; the conversion of —C(O)OH to a —NH₂ group, under Schmidt reaction conditions, or variants thereof, for example in the presence of HN₃ (which may be formed in by contacting NaN₃ with a strong acid such as H₂SO₄), or, for variants, by reaction with diphenyl phosphoryl azide ((PhO)₂P(O)N₃) in the presence of an alcohol, such as tert-butanol, which may result in the formation of a carbamate intermediate; the conversion of —C(O)NH₂ to —NH₂, for example under Hofmann rearrangement reaction conditions, for example in the presence of NaOBr (which may be formed by contacting NaOH and Br₂) which may result in the formation of a carbamate intermediate; the conversion of —C(O)N₃ (which compound itself may be prepared from the corresponding acyl hydrazide under standard diazotisation reaction conditions, e.g. in the presence of NaNO₂ and a strong acid such as H₂SO₄ or HCl) to —NH₂, for example under Curtius rearrangement reaction conditions, which may result in the formation of an intermediate isocyanate (or a carbamate if treated with an alcohol); the conversion of an alkyl carbamate to —NH₂, by hydrolysis, for example in the presence of water and base or under acidic conditions, or, when a benzyl carbamate intermediate is formed, under hydrogenation reaction conditions (e.g. catalytic hydrogenation reaction conditions in the presence of a precious metal catalyst such as Pd); halogenation of an aromatic ring, for example by an electrophilic aromatic substitution reaction in the presence of halogen atoms (e.g. chlorine, bromine, etc, or an equivalent source thereof) and, if necessary an appropriate catalyst/Lewis acid (e.g. AlCl₃ or FeCl₃).

Compounds of the invention may be isolated from their reaction mixtures using conventional techniques (e.g. recrystallisations).

It will be appreciated by those skilled in the art that, in the processes described above and hereinafter, the functional groups of intermediate compounds may need to be protected by protecting groups.

The protection and deprotection of functional groups may take place before or after a reaction in the above-mentioned schemes.

Protecting groups may be removed in accordance with techniques that are well known to those skilled in the art and as described hereinafter. For example, protected compounds/intermediates described herein may be converted chemically to unprotected compounds using standard deprotection techniques. By ‘protecting group’ we also include suitable alternative groups that are precursors to the actual group that it is desired to protect. For example, instead of a ‘standard’ amino protecting group, a nitro or azido group may be employed to effectively serve as an amino protecting group, which groups may be later converted (having served the purpose of acting as a protecting group) to the amino group, for example under standard reduction conditions described herein.

The type of chemistry involved will dictate the need, and type, of protecting groups as well as the sequence for accomplishing the synthesis.

The use of protecting groups is fully described in “Protective Groups in Organic Synthesis”, 3^{rd} edition, T. W. Greene & P. G. M. Wutz, Wiley-Interscience (1999).

### Medical and Pharmaceutical Uses

Compounds of the invention are indicated as pharmaceuticals. According to a further aspect of the invention there is provided a compound of the invention, as hereinbefore defined, for use as a pharmaceutical.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the “active” compounds to which they are metabolised) may therefore be described as “prodrugs” of compounds of the invention.

By “prodrug of a compound of the invention”, we include compounds that form a compound of the invention, in an experimentally-detectable amount, within a predetermined time (e.g. about 1 hour), following oral or parenteral administration. All prodrugs of the compounds of the invention are included within the scope of the invention.

Furthermore, certain compounds of the invention (including, but not limited to, compounds of formula I in which R³ and R⁴ are other than hydrogen so forming an ester group) may possess no or minimal pharmacological activity as such, but may be administered parenterally or orally, and thereafter be metabolised in the body to form compounds of the invention that possess pharmacological activity as such (including, but not limited to, corresponding compounds of formula I, in which R³ and R⁴ represent hydrogen). Such compounds (which also includes compounds that may possess some pharmacological activity, but that activity is appreciably lower than that of the “active” compounds of the invention to which they are metabolised), may also be described as “prodrugs”.

Thus, the compounds of the invention are useful because they possess pharmacological activity, and/or are metabolised in the body following oral or parenteral administration to form compounds which possess pharmacological activity.

Compounds of the invention may inhibit leukotriene (LT) C₄ synthase, for example as may be shown in the tests described below, and may thus be useful in the treatment of those conditions in which it is required that the formation of e.g. LTC₄, LTD₄ or LTE₄ is inhibited or decreased, or where it is required that the activation of a Cys-LT receptor (e.g. Cys-LT₁ or Cys-LT₂) is inhibited or attenuated. The compounds of the invention may also inhibit microsomal glutathione S-transferases (MGSTs), such as MGST-I, MGST-II and/or MGST-III, thereby inhibiting or decreasing the formation of LTD₄, LTE₄ or, especially, LTC₄.

Compounds of the invention may also inhibit the activity of 5-lipoxygenase-activating protein (FLAP), for example as may be shown in a test such as that described in Mol. Pharmacol., 41, 873-879 (1992). Hence, compounds of the invention may also be useful in inhibiting or decreasing the formation of LTB₄.

Compounds of the invention are thus expected to be useful in the treatment of disorders that may benefit from inhibition of production (i.e. synthesis and/or biosynthesis) of leukotrienes (such as LTC₄), for example a respiratory disorder and/or inflammation.

The term “inflammation” will be understood by those skilled in the art to include any condition characterised by a localised or a systemic protective response, which may be elicited by physical trauma, infection, chronic diseases, such as those mentioned hereinbefore, and/or chemical and/or physiological reactions to external stimuli (e.g. as part of an allergic response). Any such response, which may serve to destroy, dilute or sequester both the injurious agent and the injured tissue, may be manifest by, for example, heat, swelling, pain, redness, dilation of blood vessels and/or increased blood flow, invasion of the affected area by white blood cells, loss of function and/or any other symptoms known to be associated with inflammatory conditions.

The term “inflammation” will thus also be understood to include any inflammatory disease, disorder or condition per se, any condition that has an inflammatory component associated with it, and/or any condition characterised by inflammation as a symptom, including inter alia acute, chronic, ulcerative, specific, allergic and necrotic inflammation, and other forms of inflammation known to those skilled in the art. The term thus also includes, for the purposes of this invention, inflammatory pain, pain generally and/or fever.

Where a condition has an inflammatory component associated with it, or a condition characterised by inflammation as a symptom, the skilled person will appreciate that compounds of the invention may be useful in the treatment of the inflammatory symptoms and/or the inflammation associated with the condition.

Accordingly, compounds of the invention may be useful in the treatment of allergic disorders, asthma, childhood wheezing, chronic obstructive pulmonary disease, bronchopulmonary dysplasia, cystic fibrosis, interstitial lung disease (e.g. sarcoidosis, pulmonary fibrosis, scleroderma lung disease, and usual interstitial in pneumonia), ear nose and throat diseases (e.g. rhinitis, nasal polyposis, and otitis media), eye diseases (e.g. conjunctivitis and giant papillary conjunctivitis), skin diseases (e.g. psoriasis, dermatitis, and eczema), rheumatic diseases (e.g. rheumatoid arthritis, arthrosis, psoriasis arthritis, osteoarthritis, systemic lupus erythematosus, systemic sclerosis), vasculitis (e.g. Henoch-Schonlein purpura, Löffler's syndrome and Kawasaki disease), cardiovascular diseases (e.g. atherosclerosis), gastrointestinal diseases (e.g. eosinophilic diseases in the gastrointestinal system, inflammatory bowel disease, irritable bowel syndrome, colitis, celiaci and gastric haemorrhagia), urologic diseases (e.g. glomerulonephritis, interstitial cystitis, nephritis, nephropathy, nephrotic syndrome, hepatorenal syndrome, and nephrotoxicity), diseases of the central nervous system (e.g. cerebral ischemia, spinal cord injury, migraine, multiple sclerosis, and sleep-disordered breathing), endocrine diseases (e.g. autoimmune thyreoiditis, diabetes-related inflammation), urticaria, anaphylaxis, angioedema, oedema in Kwashiorkor, dysmenorrhoea, burn-induced oxidative injury, multiple trauma, pain, toxic oil syndrome, endotoxin chock, sepsis, bacterial infections (e.g. from Helicobacter pylori, Pseudomonas aerugiosa or Shigella dysenteriae), fungal infections (e.g. vulvovaginal candidasis), viral infections (e.g. hepatitis, meningitis, parainfluenza and respiratory syncytial virus), sickle cell anemia, hypereosinofilic syndrome, and malignancies (e.g. Hodgkins lymphoma, leukemia (e.g. eosinophil leukemia and chronic myelogenous leukemia), mastocytos, polycytemi vera, and ovarian carcinoma). In particular, compounds of the invention may be useful in treating allergic disorders, asthma, rhinitis, conjunctivitis, COPD, cystic fibrosis, dermatitis, urticaria, eosinophilic gastrointestinal diseases, inflammatory bowel disease, rheumatoid arthritis, osteoarthritis and pain.

Compounds of the invention are indicated both in the therapeutic and/or prophylactic treatment of the above-mentioned conditions.

According to a further aspect of the present invention, there is provided a method of treatment of a disease which is associated with, and/or which can be modulated by inhibition of, LTC₄ synthase and/or a method of treatment of a disease in which inhibition of the synthesis of LTC₄ is desired and/or required (e.g. respiratory disorders and/or inflammation), which method comprises administration of a therapeutically effective amount of a compound of the invention, as hereinbefore defined, to a patient suffering from, or susceptible to, such a condition.

“Patients” include mammalian (including human) patients.

The term “effective amount” refers to an amount of a compound, which confers a therapeutic effect on the treated patient. The effect may be objective (i.e. measurable by some test or marker) or subjective (i.e. the subject gives an indication of or feels an effect).

Compounds of the invention will normally be administered orally, intravenously, subcutaneously, buccally, rectally, dermally, nasally, tracheally, bronchially, sublingually, by any other parenteral route or via inhalation, in a pharmaceutically acceptable dosage form.

Compounds of the invention may be administered alone, but are preferably administered by way of known pharmaceutical formulations, including tablets, capsules or elixirs for oral administration, suppositories for rectal administration, sterile solutions or suspensions for parenteral or intramuscular administration, and the like.

Such formulations may be prepared in accordance with standard and/or accepted pharmaceutical practice.

According to a further aspect of the invention there is thus provided a pharmaceutical formulation including a compound of the invention, as herein before defined, in admixture with a pharmaceutically acceptable adjuvant, diluent or carrier.

Depending on e.g. potency and physical characteristics of the compound of the invention (i.e. active ingredient), pharmaceutical formulations that may be mentioned include those in which the active ingredient is present in at least 1% (or at least 10%, at least 30% or at least 50%) by weight. That is, the ratio of active ingredient to the other components (i.e. the addition of adjuvant, diluent and carrier) of the pharmaceutical composition is at least 1:99 (or at least 10:90, at least 30:70 or at least 50:50) by weight.

The invention further provides a process for the preparation of a pharmaceutical formulation, as hereinbefore defined, which process comprises bringing into association a compound of the invention, as hereinbefore defined, or a pharmaceutically acceptable salt thereof with a pharmaceutically-acceptable adjuvant, diluent or carrier.

Compounds of the invention may also be combined with other therapeutic agents that are useful in the treatment of a respiratory disorder (e.g. thromboxane receptor (TP) antagonists, leukotriene receptor antagonists (LTRAs), glucocorticoids, antihistamines, beta-adrenergic drugs, anticholinergic drugs and PDE₄ inhibitors and/or other therapeutic agents that are useful in the treatment of a respiratory disorder) and/or other therapeutic agents that are useful in the treatment of inflammation and disorders with an inflammatory component (e.g. NSAIDs, coxibs, corticosteroids, analgesics, inhibitors of 5-lipoxygenase, inhibitors of FLAP (5-lipoxygenase activating protein), immunosuppressants and sulphasalazine and related compounds and/or other therapeutic agents that are useful in the treatment of inflammation).

According to a further aspect of the invention, there is provided a combination product comprising:

(A) a compound of the invention, as hereinbefore defined; and
(B) another therapeutic agent that is useful in the treatment of a respiratory disorder and/or inflammation,
wherein each of components (A) and (B) is formulated in admixture with a pharmaceutically-acceptable adjuvant, diluent or carrier.

Such combination products provide for the administration of a compound of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

Thus, there is further provided:

(1) a pharmaceutical formulation including a compound of the invention, as hereinbefore defined, another therapeutic agent that is useful in the treatment of a respiratory disorder and/or inflammation, and a pharmaceutically-acceptable adjuvant, diluent or carrier; and
(2) a kit of parts comprising components:
(a) a pharmaceutical formulation including a compound of the invention, as hereinbefore defined, in admixture with a pharmaceutically-acceptable adjuvant, diluent or carrier; and
(b) a pharmaceutical formulation including another therapeutic agent that is useful in the treatment of a respiratory disorder and/or inflammation in admixture with a pharmaceutically-acceptable adjuvant, diluent or carrier,
   which components (a) and (b) are each provided in a form that is suitable for administration in conjunction with the other.

The invention further provides a process for the preparation of a combination product as hereinbefore defined, which process comprises bringing into association a compound of the invention, as hereinbefore defined, or a pharmaceutically acceptable salt thereof with the other therapeutic agent that is useful in the treatment of a respiratory disorder and/or inflammation, and at least one pharmaceutically-acceptable adjuvant, diluent or carrier.

By “bringing into association”, we mean that the two components are rendered suitable for administration in conjunction with each other.

Thus, in relation to the process for the preparation of a kit of parts as hereinbefore defined, by bringing the two components “into association with” each other, we include that the two components of the kit of parts may be:

(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Compounds of the invention may be administered at varying doses. Oral, pulmonary and topical dosages may range from between about 0.01 mg/kg of body weight per day (mg/kg/day) to about 100 mg/kg/day, preferably about 0.01 to about 10 mg/kg/day, and more preferably about 0.1 to about 5.0 mg/kg/day. For e.g. oral administration, the compositions typically contain between about 0.01 mg to about 500 mg, and preferably between about 1 mg to about 100 mg, of the active ingredient. Intravenously, the most preferred doses will range from about 0.001 to about 10 mg/kg/hour during constant rate infusion. Advantageously, compounds may be administered in a single daily dose, or the total daily dosage may be administered in divided doses of two, three or four times daily.

In any event, the physician, or the skilled person, will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. The above-mentioned dosages are exemplary of the average case; there can, of course, be individual instances where higher or lower dosage ranges are merited, and such are within the scope of this invention.

Compounds of the invention may have the advantage that they are effective inhibitors of LTC₄ synthase.

Compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties over, compounds known in the prior art, whether for use in the above-stated indications or otherwise. In particular, compounds of the invention may have the advantage that they are more efficacious and/or exhibit advantageous properties in vivo.

### Biological Test

### In Vitro Assay

### Method A

In the assay, LTC₄ synthase catalyses the reaction where the substrate LTA₄ methyl ester is converted to the corresponding LTC₄ methyl ester. Recombinant human LTC₄ synthase is expressed in Piccia pastoralis and the purified enzyme is dissolved in 25 mM Tris-buffer pH 7.8 and stored at −20° C. The assay is performed in phosphate buffered saline (PBS) pH 7.4, supplemented with 5 mM glutathione (GSH). The reaction is terminated by addition of MeCN/MeOH/acetic acid (50/50/1). The assay is performed at rt in 96-well plates. Analysis of the formed LTC₄ methyl ester is performed with reversed phase HPLC (Waters 2795 utilizing an Onyx Monolithic C18 column). The mobile phase consists of MeCN/MeOH/H₂O (32.5/30/37.5) with 1% acetic acid, pH adjusted with NH₃ to 5.6, and absorbance measured at 280 nm with a Waters 2487 UV-detector.

The following is added chronologically to each well:
1. 50 μL assay buffer, PBS with 5 mM GSH.
2. 0.5 μL inhibitor in DMSO.
3. 2 μL LTC₄ synthase in PBS. The total protein concentration in this solution is 0.025 mg/mL. Incubation of the plate at rt for 10 min.
4. 1-1.5 μL LTA₄ methyl ester (final conc. 10 μM). Incubation of the plate at rt for 1 min.
5. 50 μL stop solution.
   80 μL of the incubation mixture is analysed with HPLC.

### Method B

### In Vitro Assay

In the assay, LTC₄ synthase catalyses the reaction where the substrate LTA₄ is converted to LTC₄. Recombinant human LTC₄ synthase is expressed in Piccia pastoralis and the purified enzyme is dissolved in 25 mM Tris-buffer pH 7.8 and stored at −20° C. The assay is performed in phosphate buffered saline (PBS) pH 7.4, supplemented with 5 mM glutathione (GSH). The assay is performed at rt in 384-well plates. Analysis of the formed LTC₄ is performed using costume made homogenous time-resolved fluorescence (HTRF) detection (Cisbio).

The following is added chronologically to each well:
1. 50 μL assay buffer, PBS with 5 mM GSH.
2. 0.5 μL inhibitor in DMSO.
3. 2 μl LTC₄ synthase in PBS. The total protein concentration in this solution is 0.025 mg/ml. Incubation of the plate at rt for 10 min.
4. 1-1.5 μL LTA₄ (final conc. 2.5 μM). Incubation of the plate at rt for 1 min.
   10 μL of the incubation mixture is analysed using HTRF detection.

### In Vivo Assay

### Mouse Peritoneal Inflammation Model

The in vivo efficacy of leukotriene biosynthesis inhibitors was assed by using a model of peritoneal inflammation. Male BALB/c mice (weighing 20-25 grams) received an intraperitoneal injection (ip) of 0.5 ml zymosan dissolved in phosphate buffer saline (PBS) (pH 7.5, 1 mg/mL). 5 Minutes before the zymosan injection compounds (1-10 mg/kg) or vehicle were administered ip (10 mL/kg). 30 min after the zymosan injection the mice were anaesthetized and the peritoneal cavity was flushed with 4 mL PBS. The peritoneal lavage was centrifuged at 6000 rcf for 5 minutes and the cysteinyl leukotrienes concentration in the supernatant was determined by EIA. The cysteinyl leukotrienes concentration in the peritoneal lavage from compound treated mice was compared to vehicle treated mice and the % inhibition was calculated.

The invention is illustrated by way of the following examples, in which the following abbreviations may be employed:

aq aqueous
brine saturated aqueous solution of NaCl
DCM dichloromethane
DMF dimethylformamide
DEAD diethyl azodicarboxylate
EtOAc ethyl acetate
Eq equivalent
MeCN acetonitrile
MeOH methanol
NMR nuclear magnetic resonance
Pd/C palladium on charcoal
Pd₂(dba)₃ tris(dibenzylideneacetone)dipalladium(0)
rt room temperature
rx reflux temperature
sat saturated
TEA triethylamine
THF tetrahydrofuran
xantphos 9,9-dimethyl-4,5-bis(diphenylphosphino)-9H-xanthene

### EXAMPLE 1:1

### 1-(4-Benzyloxyphenyl)-2-carboxymethyl-5-(4-isopropoxyphenoxy)indole-3-carboxylic acid

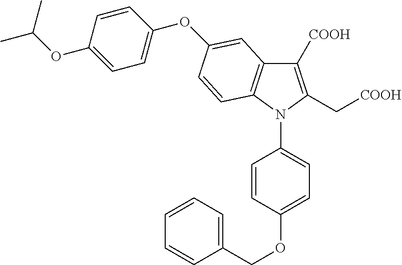

### (a) 1-(4-Benzyloxyphenyl)-5-hydroxy-2-methoxycarbonylmethylindole-3-carboxylic acid methyl ester

A catalytic amount of p-toluenesulphonic acid was added to a mixture of 4-benzyloxyaniline (10.9 g, 54.7 mmol), 1,3-acetonedicarboxylic acid dimethyl ester (8.1 mL, 54.7 mmol) and then dissolved in CHCl₃. The mixture was heated at rx for 6.5 h and concentrated. 1,4-Benzoquinone (7.0 g, 64.8 mmol) and MeCN (110 mL) was added and the mixture was stirred at 70° C. for 2 d and kept at 4° C. for 2 d. The precipitate was collected and recrystallized from MeCN to give the sub-title compound. Yield: 4.2 g (14%).

### (b) 1-(4-Benzyloxyphenyl)-5-(4-isopropoxyphenoxy)-2-methoxycarbonylmethylindole-3-carboxylic acid methyl ester

A mixture of 1-(4-benzyloxyphenyl)-5-hydroxy-2-methoxycarbonyl methylindole-3-carboxylic acid methyl ester (3.0 g, 6.3 mmol), 4-isopropoxyphenylboronic acid (2.84 g, 15.8 mmol), Cu(OAc)₂ (1.14 g, 6.3 mmol), pyridine (1.29 mL), TEA (2.2 mL) and DCM (200 mL) was stirred at rt for 2 d. The mixture was filtered and concentrated, and the residue was purified by chromatography to give the sub-title compound.

### (c) 1-(4-Benzyloxyphenyl)-2-carboxymethyl-5-(4-isopropoxyphenoxy)indole-3-carboxylic acid

A mixture of 1-(4-benzyloxyphenyl)-5-(4-isopropoxyphenoxy)-2-methoxycarbonylmethylindole-3-carboxylic acid methyl ester (0.3 mmol, 0.17 g), NaOH (2M, aq, 3 mL) and dioxane (3 mL) was stirred at 70° C. for 20 h, cooled and neutralized with HCl (1M, aq). Extractive workup (EtOAc, brine), drying (Na₂SO₄) and concentration gave a material that was treated with DCM. The solid material was collected and dried to give the title compound. Yield: 0.12 g (75%).

¹H NMR (DMSO-d₆) δ: 12.2-12.6 (br s, 2H) 7.61 (unresolved d; 1H) 7.19-7.58 (m, 9H) 6.84-7.02 (m, 6H) 5.20 (s, 2H) 4.52 (heptet, J=6.1 Hz, 1H) 3.94 (s, 2H) 1.25 (d, J=6.1 Hz, 6H).

### EXAMPLE 1:2

### 2-Carboxymethyl-5-(4-isopropoxyphenoxy)-1-(4-(4-methylbenzyloxy)phenyl)indole-3-carboxylic acid

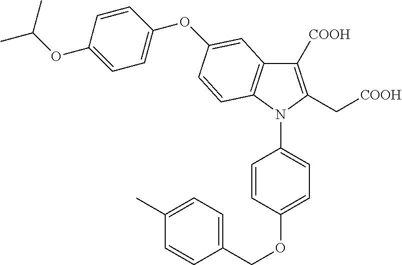

### (a) 1-(4-Hydroxyphenyl)-5-(4-isopropoxyphenoxy)-2-methoxycarbonylmethylindole-3-carboxylic acid methyl ester

A mixture of 1-(4-benzyloxyphenyl)-5-(4-isopropoxyphenoxy)-2-methoxycarbonylmethylindole-3-carboxylic acid methyl ester (2.26 g, 3.9 mmol, see Example 1:1, step (b)), Pd/C (0.55 g), MeOH (40 mL) and EtOAc (10 mL) was hydrogenated at ambient temperature and pressure for 20 h. The mixture was filtered through Celite and concentrated to give the sub-title compound.

### (b) 5-(4-Isopropoxyphenoxy)-1-(4-(4-methylbenzyloxy)phenyl)-2-methoxycarbonylmethylindole-3-carboxylic acid methyl ester

A mixture of 1-(4-hydroxyphenyl)-5-(4-isopropoxyphenoxy)-2-methoxycarbonylmethylindole-3-carboxylic acid methyl ester (196 mg, 0.40 mmol), 4-methylbenzyl bromide (89 mg, 0.44 mmol), K₂CO₃ (120 mg, 0.88 mmol) and DMF (3 mL) was stirred at 70° C. for 20 h. Extractive workup (EtOAc, water, brine), drying (Na₂SO₄), concentration and purification by chromatography gave the sub-title compound. Yield: 190 mg (79%).

### (c) 2-Carboxymethyl-5-(4-isopropoxyphenoxy)-1-(4-(4-methylbenzyloxy)-phenyl)indole-3-carboxylic acid

The title compound was prepared from 5-(4-isopropoxyphenoxy)-1-(4-(4-methylbenzyloxy)phenyl)-2-methoxycarbonylmethylindole-3-carboxylic acid methyl ester in accordance with Example 1:1, step (c).

¹H NMR (DMSO-d₆) δ: 12.7-12.2 (2H, br s) 7.61 (1H, d, J=2.1 Hz) 7.44-7.30 (4H, m) 7.29-7.16 (4H, m) 7.00-6.84 (6H, m) 5.14 (2H, s) 4.52 (1H, septet, J=6.1 Hz) 3.94 (2H, s) 2.36 (3H, s) 1.25 (6H, d, J=6.1 Hz)

### EXAMPLES 1:3-1:11

The title compounds were synthesized from 1-(4-hydroxyphenyl)-5-(4-isopropoxyphenoxy)-2-methoxycarbonylmethylindole-3-carboxylic acid methyl ester and the appropriate arylmethyl halide in accordance with Example 1:2, see Table 1.

TABLE 1

| Ex- | Chemical structure |
|---|---|
| am- | Name |
| ple | ¹H-NMR (DMSO-d₆, δ) |
| 1:3 | 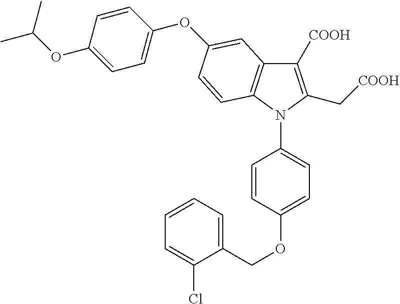 |
| | 2-Carboxymethyl-1-[4-(2-chlorobenzyloxy)phenyl]-5-(4-isopropoxy- |
| | phenoxy)indole-3-carboxylic acid |
| | 12.6-12.3 (2H, br s) 7.74-7.50 (3H, m) 7.49-7.20 (6H, m) 7.03-6.82 |
| | (6H, m) 5.26 (2H, s) 4.52 (1H, septet, J = 6.1 Hz) 3.95 (2H, s) 1.25 |
| | (6H, d, J = 6.1 Hz) |
| 1:4 | 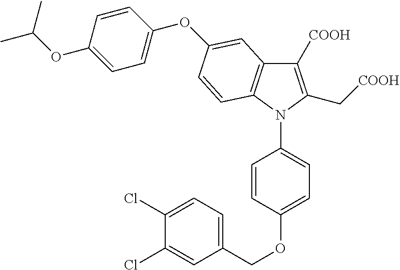 |
| | 2-Carboxymethyl-1-[4-(3,4-dichlorobenzyloxy)phenyl]-5-(4- |
| | isopropoxyphenoxy)indole-3-carboxylic acid |
| | 12.6-12.3 (2H, br s) 7.76-7.58 (3H, m) 7.53-7.21 (5H, m) 7.04-6.82 |
| | (6H, m) 5.30 (2H, s) 4.52 (1H, septet, J = 6.1 Hz) 3.96 (2H, s) 1.25 |
| | (6H, d, J = 6.1 Hz) |
| 1:5 | 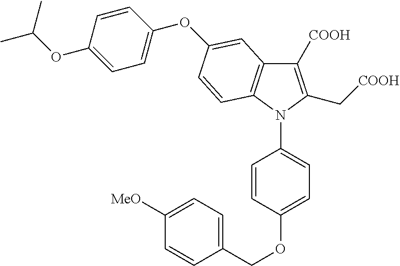 |
| | 2-Carboxymethyl-1-5-(4-isopropoxyphenoxy)-[4-(4- |
| | methoxybenzyloxy)-phenyl]indole-3-carboxylic acid |
| | 12.5-12.3 (2H, br s) 7.62 (1H, d, J = 2.1 Hz) 7.50-7.29 (4H, m) |
| | 7.28-7.17 (2H, m) 7.04-6.83 (8H, m) 5.10 (2H, s) 4.52 (1H, septet, |
| | J = 6.1 Hz) 3.92 (2H, s) 3.78 (3H, s) 1.25 (6H, d, J = 6.1 Hz) |
| 1:6 | 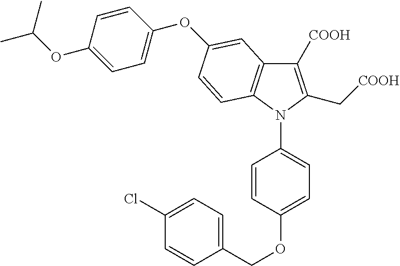 |
| | 2-Carboxymethyl-1-[4-(4-chlorobenzyloxy)phenyl]-5-(4-isopropoxy- |
| | phenoxy)indole-3-carboxylic acid |
| | 12.5-12.3 (2H, br s) 7.62 (1H, d, J = 2.1 Hz) 7.59-7.44 (4H, m) |
| | 7.41-7.31 (2H, m) 7.30-7.18 (2H, m) 7.01-6.82 (6H, m) 5.20 (2H, s) |
| | 4.52 (1H, septet, J = 6.1 Hz) 3.95 (2H, s) 1.25 (6H, d, J = 6.1 Hz) |
| 1:7 | 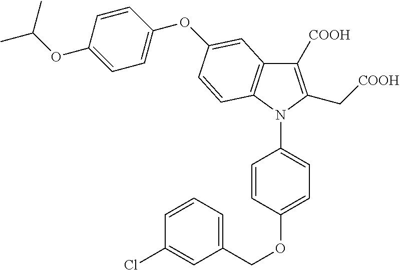 |
| | 2-Carboxymethyl-1-[4-(3-chlorobenzyloxy)phenyl]-5-(4-isopropoxy- |
| | phenoxy)indole-3-carboxylic acid |
| | 12.6-12.3 (2H, br s) 7.67-7.19 (9H, m) 7.07-6.78 (6H, m) 5.22 (2H, s) |
| | 4.52 (1H, septet, J = 6.1 Hz) 3.95 (2H, s) 1.25 (6H, d, J = 6.1 Hz) |
| 1:8 | 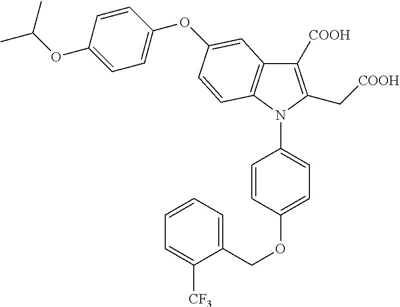 |
| | 2-Carboxymethyl-1-5-(4-isopropoxyphenoxy)-[4-(2-trifluoromethyl- |
| | benzyloxy)phenyl]indole-3-carboxylic acid |
| | 12.6-12.3 (2H, br s) 7.91-7.72 (3H, m) 7.70-7.56 (2H, m) 7.45-7.33 |
| | (2H, m) 7.32-7.21 (2H, m) 7.05-6.82 (6H, m) 5.33 (2H, s) 4.52 (1H, |
| | septet, J = 6.0 Hz) 3.96 (2H, s) 1.25 (6H, d, J = 6.0 Hz) |
| 1:9 | 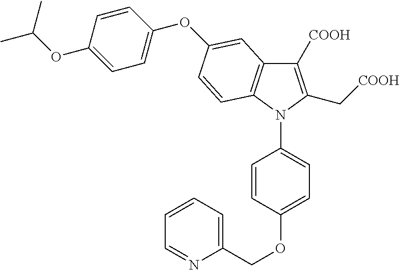 |
| | 2-Carboxymethyl-1-5-(4-isopropoxyphenoxy)-[4-(2- |
| | pyridinylmethoxy)-phenyl]indole-3-carboxylic acid |
| | 12.6-12.3 (2H, br s) 8.61 (1H, dd, J = 2.0 and 0.9 Hz) 7.95-7.83 (1H, |
| | m) 7.72-7.54 (2H, m) 7.46-7.21 (5H, m) 7.02-6.83 (6H, m) 5.28 (2H, |
| | s) 4.52 (1H, septet, J = 5.9 Hz) 3.95 (s, 2H) 1.25 (6H, d, J = 5.9 Hz) |
| 1:10 | 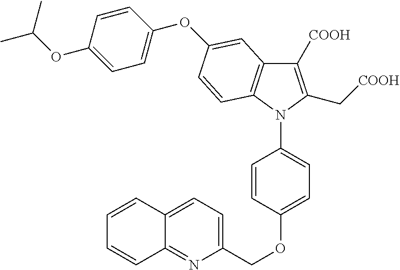 |
| | 2-Carboxymethyl-1-5-(4-isopropoxyphenoxy)-[4-(2- |
| | quinolinylmethoxy)-phenyl]indole-3-carboxylic acid |
| | 12.9-12.0 (2H, br s) 8.47 (1H, d, J = 8.6 Hz) 8.11-7.99 (2H, m) |
| | 7.86-7.76 (1H, m) 7.77 (1H, d, J = 8.6 Hz) 7.70-7.59 (2H, m) 7.43- |
| | 7.27 (4H, m) 6.99-6.84 (6H, m) 5.48 (2H, s) 4.52 (1H, septet, J = |
| | 5.9 Hz) 3.95 (2H, s) 1.25 (6H, d, J = 5.9 Hz) |
| 1:11 | 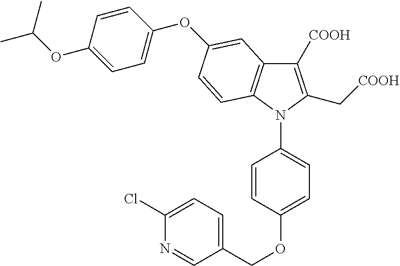 |
| | 2-Carboxymethyl-1-[4-(6-chloro-3-pyridinylmethoxy)phenyl]-5-(4- |
| | isopropoxyphenoxy)indole-3-carboxylic acid |
| | 12.7-12.1 (2H, br s) 8.58 (1H, d, J = 2.5 Hz) 8.02 (1H, dd, J = 8.3 |
| | and 2.5 Hz) 7.65-7.62 (1H, m) 7.59 (1H, d, J = 4.5 Hz) 7.44-7.21 |
| | (4H, m) 6.99-6.84 (6H, m) 5.26 (2H, s) 4.52 (1H, septet, J = 6.0 |
| | Hz) 3.94 (2H, s) 1.25 (6H, d, J = 6.0 Hz) |

### EXAMPLE 2:1

### 1-(4-Benzyloxyphenyl)-2-carboxymethyl-5-(4-trifluoromethoxyphenoxy)indole-3-carboxylic acid

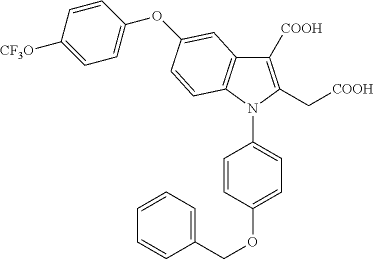

The title compound was prepared from 1-(4-benzyloxyphenyl)-5-hydroxy-2-methoxycarbonylmethylindole-3-carboxylic acid methyl ester and 4-trifluoromethoxyphenylboronic acid in accordance with Example 1:1, steps (b) and (c).

¹H NMR (DMSO-d₆) δ: 12.6-12.3 (2H, br s) 7.73 (1H, d, J=2.1 Hz) 7.57-7.20 (11H, m) 7.12-6.92 (4H, m) 5.20 (2H, s) 3.97 (2H, s).

### EXAMPLE 3:1

### 2-Carboxymethyl-5-(4-isopropoxyphenoxy)-1-[4-(4-trifluoromethylthiophenoxy)-phenyl]indole-3-carboxylic acid

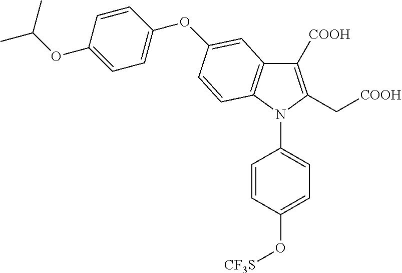

A mixture of 1-(4-hydroxyphenyl)-5-(4-isopropoxyphenoxy)-2-methoxycarbonylmethylindole-3-carboxylic acid methyl ester (0.24 g, 0.49 mmol, see Example 1:2, step (a)), 4-(trifluoromethylthio)-iodobenzene (223 mg, 0.74 mmol), CuI (4.6 mg, 0.025 mmol), N,N′-dimethylglycine hydrochloride (13.6 mg, 0.098 mmol), Cs₂CO₃ (319 mg, 0.92 mmol) and dry dioxane (5 mL) was stirred at 100° C. for 2 d. The mixture was filtered through Celite, concentrated and purified by chromatography to give a diester which was hydrolyzed with aq NaOH in accordance with Example 1:2, step (c) to give the title compound. Yield: 0.12 g (38%).

¹H NMR (DMSO-d₆) δ: 12.6-12.3 (2H, br s) 7.84-7.73 (2H, m) 7.62 (1H, d, J=2.2 Hz) 7.56-7.46 (2H, m) 7.42-7.33 (2H, m) 7.31-7.22 (2H, m) 7.07 (1H, d, J=8.9 Hz) 6.99-6.86 (5H, m) 4.01 (2H, s) 4.53 (1H, septet, J=6.0 Hz) 1.25 (6H, d, J=6.0 Hz).

### EXAMPLES 3:2-3:5

The title compounds were synthesized from 1-(4-hydroxyphenyl)-5-(4-isopropoxyphenoxy)-2-methoxycarbonylmethylindole-3-carboxylic acid methyl ester (see Example 1:1, step (b)) and the appropriate aryl boronic acid in accordance with Example 3:1; see Table 2.

TABLE 2

| Ex- | Chemical structure |
|---|---|
| am- | Name |
| ple | ¹H-NMR (DMSO-d₆, δ) |
| 3:2 | 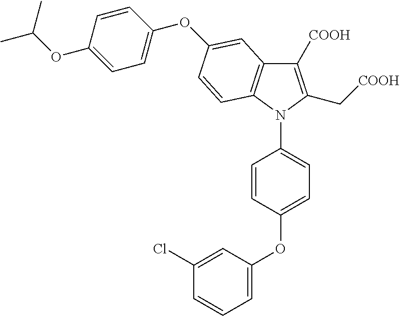 |
| | 2-Carboxymethyl-1-[4-(3-chlorophenoxy)phenyl]-5-(4-isopropoxy- |
| | phenoxy)indole-3-carboxylic acid |
| | 12.6-12.3 (2H, br s) 7.61 (1H, d, J = 2.0 Hz) 7.53-7.42 (3H, m) 7.33- |
| | 7.23 (4H, m) 7.17-7.08 (1H, m) 7.04 (1H, d, J = 8.8 Hz) 6.98-6.85 |
| | (5H, m) 4.52 (1H, septet, J = 5.9 Hz) 4.00 (2H, s) 1.25 (6H, d, J = |
| | 5.9 Hz) |
| 3:3 | 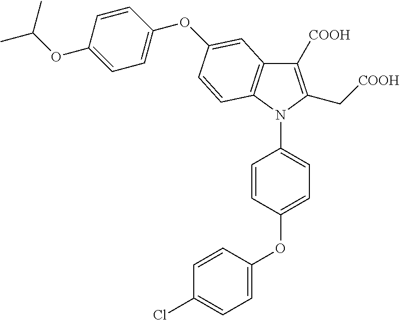 |
| | 2-Carboxymethyl-1-[4-(4-chlorophenoxy)phenyl]-5-(4-isopropoxy- |
| | phenoxy)indole-3-carboxylic acid |
| | 7.61 (1H, d, J = 2.0 Hz), 7.55-7.41 (4H, m) 7.29-7.16 (4H, m) 7.04- |
| | 6.83 (6H, m) 4.52 (1H, septet, J = 5.9 Hz) 3.74 (2H, s) 1.25 (6H, d, |
| | J = 5.9 Hz) |
| 3:4 | 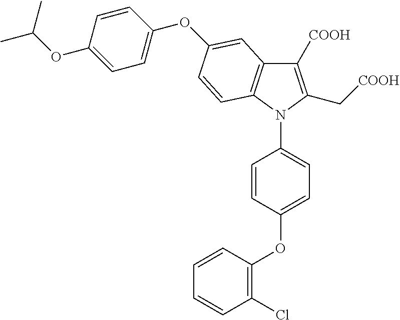 |
| | 2-Carboxymethyl-1-[4-(2-chlorophenoxy)phenyl]-5-(4-isopropoxy- |
| | phenoxy)indole-3-carboxylic acid |
| | 12.6-12.3 (2H, br s) 7.71-7.59 (2H, m) 7.52-7.39 (3H, m) 7.36-7.24 |
| | (2H, m) 7.22-7.12 (2H, m) 7.01 (1H, d, J = 8.8 Hz) 6.97-6.84 (5H, |
| | m) 4.52 (1H, septet, J = 5.9 Hz) 3.98 (2H, s) 1.25 (6H, d, J = 5.9 Hz) |
| 3:5 | 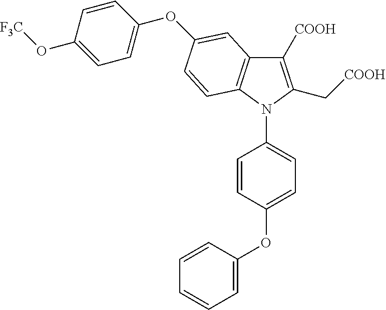 |
| | 2-Carboxymethyl-5-(4-isopropoxyphenoxy)-1-[4-(4- |
| | trifluoromethoxyphenoxy)phenyl]indole-3-carboxylic acid |
| | 12.4-12.7 (br s, 2H), 7.74 (d, J = 2.0 Hz, 1H) 7.41-7.53 (m, 4H) |
| | 7.30-7.39 (m, 2H) 7.03-7.29 (m, 8H) 6.99 (dd, J = 8.8 and 2.0 |
| | Hz, 1H) 4.02 (s, 2H) |

### EXAMPLES 4:1-4:5

The title compounds were synthesized in accordance with Example 1:1 from an appropriately substituted aniline and 1,3-acetonedicarboxylic acid dimethyl or diethyl ester, followed by the reaction with an appropriately substituted arylboronic acid and hydrolysis; see Table 3.

TABLE 3

| Ex- | Chemical structure |
|---|---|
| am- | Name |
| ple | ¹H-NMR (DMSO-d₆, δ) |
| 4:1 | 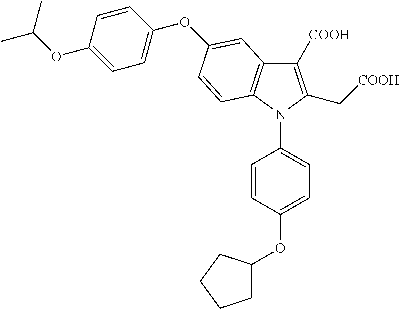 |
| | 2-Carboxymethyl-1-(4-cyclopentyloxyphenyl)-5- |
| | (4-isopropoxyphenoxy)-indole-3-carboxylic acid |
| | 12.3-12.6 (br s, 2H) 7.61 (d, J = 2.1 Hz, 1H) 7.23-7.38 (m, 2H) |
| | 7.04-7.17 (m, 2H) 6.79-7.02 (m, 6H) 4.85-4.99 (m, 1H) 4.52 (heptet, |
| | J = 6.1 Hz, 1H) 3.94 (s, 2H) 1.51-2.10 (m, 8H) 1.25 (d, J = 6.1 Hz, |
| | 6H) |
| 4:2 | 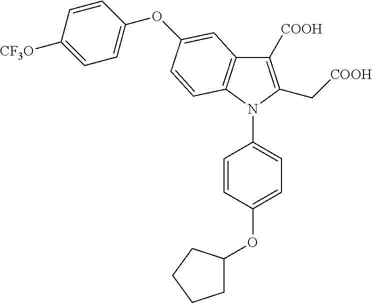 |
| | 2-Carboxymethyl-1-(4-cyclopentyloxyphenyl)-5-(4- |
| | trifluoromethoxyphenoxy)indole-3-carboxylic acid |
| | 12.3-12.5 (br s, 2H) 7.72 (unresolved d, 1H) 7.24-7.44 (m, 4H) |
| | 6.87-7.22 (m, 6H) 4.83-5.02 (m, 1H) 3.96 (s, 2H) 1.50-2.14 (m, |
| | 8H) |
| 4:3 | 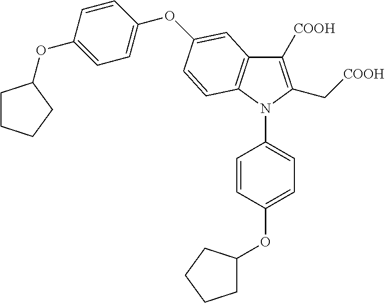 |
| | 2-Carboxymethyl-5-(4-cyclopentyloxyphenoxy)-1-(4- |
| | cyclopentyloxyphenyl)indole-3-carboxylic acid |
| | 12.6-12.3 (2H, br s) 7.60 (1H, d, J = 2.1 Hz) 7.36-7.24 (2H, m) |
| | 7.17-7.05 (2H, m) 7.01-6.78 (6H, m) 4.97-4.85 (1H, m) 4.81-4.69 |
| | (1H, m) 3.96 (2H, s) 2.16-1.39 (16H, m) |
| 4:4 | 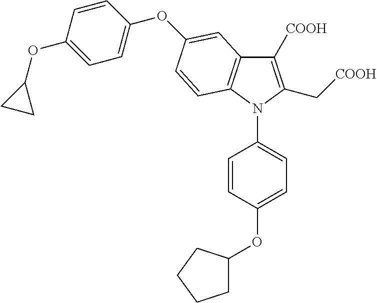 |
| | 2-Carboxymethyl-1-(4-cyclopentyloxyphenyl)-5-(4- |
| | cyclopropoxyphenoxy)-indole-3-carboxylic acid |
| | 12.6-12.3 (2H, br s) 7.61 (1H, d, J = 2.1 Hz) 7.36-7.26 (2H, m) |
| | 7.18-6.92 (7H, m) 6.88 (1H, dd, J = 8.7 and 2.1 Hz) 4.98-4.86 |
| | (1H, m) 3.94 (2H, s) 3.86-3.74 (1H, m) 2.08-1.52 (8H, m) |
| | 0.83-0.58 (4H, m) |
| 4:5 | 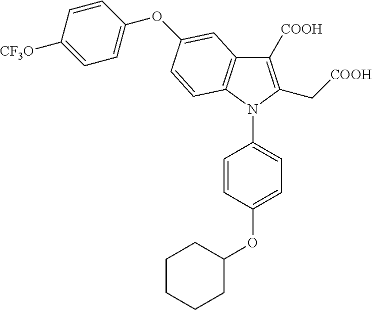 |
| | 2-Carboxymethyl-1-(4-cyclohexyloxyphenyl)-5-(4- |
| | trifluoromethoxyphenoxy)indole-3-carboxylic acid |
| | 12.3-12.6 (br s, 2H) 7.72 (d, J = 2.2 Hz, 1H) 7.26-7.42 (m, 4H) |
| | 7.11-7.22 (m, 2H) 7.00-7.10 (m, 3H) 6.96 (dd, J = 8.8 and 2.2 Hz, |
| | 1H) 4.37-4.55 (m, 1H) 3.96 (s, 2H) 1.89-2.12 (m, 2H) 1.66-1.87 |
| | (m, 2H) 1.12-1.65 (m, 6H) |

### EXAMPLE 5:1

### 2-Carboxymethyl-1-[4-(4,4-dimethylcyclohexyloxy)phenyl]-5-(4-isopropoxyphenoxy)indole-3-carboxylic acid

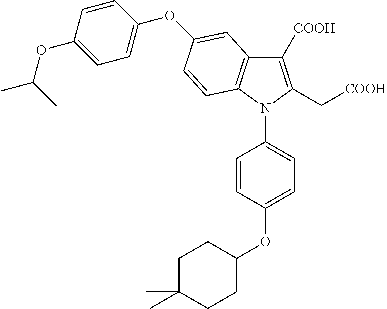

4,4-Dimethylcyclohexanol (65 mg, 0.510 mmol), triphenylphosphine (134 mg, 0.510 mmol) and DEAD (89 mg, 0.510 mmol) was added to a mixture of 1-(4-hydroxyphenyl)-5-(4-isopropoxyphenoxy)-2-methoxycarbonylmethylindole-3-carboxylic acid methyl ester (125 mg, 0.255 mmol, (see Example 1:2, step (a)) and THF (4 mL). The mixture was stirred at rt overnight. Extractive workup (EtOAc, water), drying (Na₂SO₄), concentration and purification by chromatography, followed by hydrolysis in accordance with Example 1:1, step (c) gave the title compound. Yield: 57 mg (39%).

¹H NMR (DMSO-d₆) δ: 12.38 (br s, 2H) 7.61 (d, J=2.4 Hz, 1H) 7.34-7.27 (m, 2H) 7.19-7.11 (m, 2H) 6.99-6.86 (m, 6H) 4.52 (septet, J=6 Hz, 1H) 4.47-4.38 (m, 1H) 3.94 (s, 2H), 1.93-1.82 (m, 2H) 1.69-1.57 (m, 2H) 1.52-1.42 (m, 2H) 1.37-1.28 (m, 2H) 1.25 (d, J=6 Hz, 6H) 0.96 (s, 3H) 0.94 (s, 3H)

### EXAMPLES 5:2-5:3

The title compounds were synthesized from 1-(4-hydroxyphenyl)-5-(4-isopropoxyphenoxy)-2-methoxycarbonylmethylindole-3-carboxylic acid methyl ester and the appropriate alcohol in accordance with Example 5:1; see Table 4.

TABLE 4

| | Chemical structure |
|---|---|
| | Name |
| Example | ¹H-NMR (DMSO-d₆, δ) |
| 5:2 | 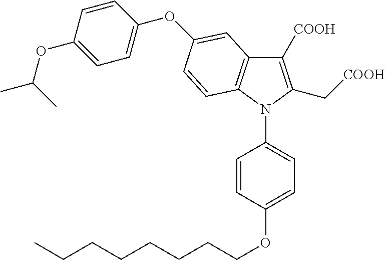 |
| | 2-Carboxymethyl-1-(4-octyloxyphenyl)-5-(4-isopropoxyphenoxy)indole-3- |
| | carboxylic acid |
| | 12.38 (br s, 2H) 7.61 (d, J = 2.4 Hz, 1H) 7.36-7.27 (m, 2H) 7.18-7.11 (m, |
| | 2H) 6.99-6.85 (m, 6H) 4.52 (septet, J = 6 Hz, 1H) 4.05 (t, J = 6.4 Hz, 2H) |
| | 3.94 (s, 2H) 1.82-1.69 (m, 2H) 1.51-1.40 (m, 2H) 1.39-1.20 (m, 8H) 1.25 |
| | (d, J = 6 Hz, 6H) 0.87 (t, J = 6.7 Hz, 3H) |
| 5:3 | 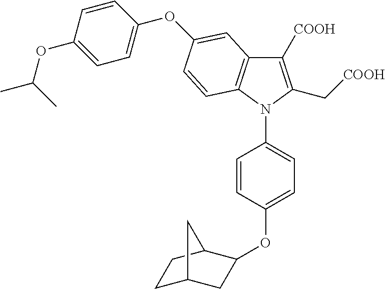 |
| | 2-Carboxymethyl-5-(4-isopropoxyphenoxy)-1-[4-(2-norbomanyloxy)- |
| | phenyl]indole-3-carboxylic acid |
| | 12.38 (br s, 2H) 7.61 (d, J = 2.4 Hz, 1H) 7.35-7.28 (m, 2H) 7.20-7.12 (m, 2H) |
| | 6.98-6.86 (m, 6H),4.52 (septet, J = 6 Hz, 1H) 4.14-3.73 (m, 2H) 3.94 (s, 2H) 2.40-2.16 |
| | (m, 3H) 1.98-1.71 (m, 1H) 1.59-1.08 (m, 6H) 1.25 (d, J = 6 Hz, 6H) 0.86-0.74 (m, 1H) |

### EXAMPLE 6:1

### 2-Carboxymethyl-1-[4-(3-pyridinylmethylamino)phenyl]-5-(4-trifluoromethoxyphenoxy)indole-3-carboxylic acid

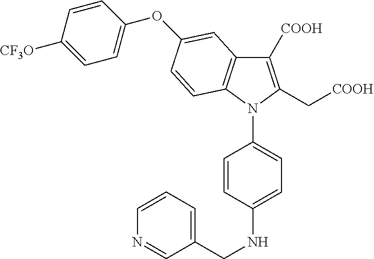

### (a) 2-Methoxycarbonylmethyl-5-(4-trifluoromethoxyphenoxy)-1-(4-trifluoromethylsulfonyloxyphenyl)indole-3-carboxylic acid methyl ester

A mixture of 2-carboxymethyl-1-(4-hydroxyphenyl)-5-(4-trifluoromethoxyphenoxy)indole-3-carboxylic acid methyl ester (8.9 g, 17.3 mmol, prepared via debenzylation of 1-(4-benzyloxyphenyl)-2-carboxymethyl-5-(4-trifluoromethoxyphenoxy)indole-3-carboxylic acid methyl ester (see Example 2:1) in accordance with Example 1:2, step (a)), triflic anhydride (3 mL), pyridine (3.5 mL) and DCM (300 mL) was stirred at 0° C. for 45 min and allowed to attain rt. Extractive workup (citric acid (aq, 10%), brine), drying (Na₂SO₄), concentration and chromatographic purification gave the sub-title compound. Yield: 10.1 g (90%).

### (b) 2-Carboxymethyl-1-[4-(3-pyridinylmethylamino)phenyl]-5-(4-trifluoromethoxyphenoxy)indole-3-carboxylic acid

A mixture of 2-methoxycarbonylmethyl-5-(4-trifluoromethoxyphenoxy)-1-(4-trifluoromethylsulfonyloxyphenyl)indole-3-carboxylic acid methyl ester (250 mg, 0.39 mmol), 3-pyridylmethylamine (63 mg, 0.58 mmol), Pd₂(dba)₃ (10 mg, 0.011 mmol), xantphos (26 mg, 0.044 mmol), Cs₂CO₃ (127 mg, 0.54 mmol) and toluene (5 mL) was stirred at 80° C. overnight in a sealed tube. The mixture was allowed to cool, filtered through silica gel, concentrated and purified by chromatography to give a diester which was hydrolyzed with aq NaOH in accordance with Example 1:2, step (c) to give the title compound. Yield: 122 mg (55%).

### EXAMPLES 6:2-6:9

The title compounds were synthesized from 2-methoxycarbonylmethyl-5-(4-trifluoromethoxyphenoxy)-1-(4-trifluoromethylsulfonyloxyphenyl)indole-3-carboxylic acid methyl ester and the appropriate amine in accordance with Example 6:2; see Table 5.

¹H NMR (DMSO-d₆) δ: 12.40 (br s, 2H) 8.64 (d, J=1.6 Hz, 1H) 8.48 (dd, J=4.8 and 1.6 Hz, 1H) 7.81 (dt, J=7.8 and 1.8 Hz, 1H) 7.70 (d, J=2.4 Hz, 1H) 7.39 (ddd, J=7.8, 4.8 and 0.6 Hz, 1H) 7.36-7.30 (m, 2H) 7.14-7.08 (m, 2H) 7.07-7.02 (m, 2H) 7.01-6.97 (m, 1H) 6.94 (dd, J=8.8 and 2.4 Hz, 1H) 6.82-6.73 (m, 3H) 4.38 (d, J=5.6 Hz, 2H) 3.93 (s, 2H).

TABLE 5

| | Chemical structure |
|---|---|
| | Name |
| Example | ¹H-NMR (DMSO-d₆, δ) |
| 6:2 | 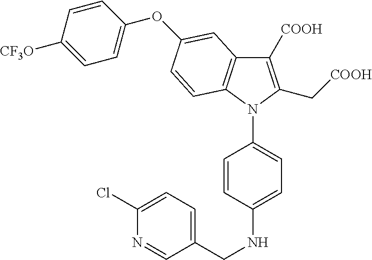 |
| | 2-Carboxymethyl-1-{4-[(6-chloro-3-pyridinylmethyl)amino]phenyl}-5- |
| | (4-trifluoromethoxyphenoxy)indole-3-carboxylic acid |
| | 12.40 (br s, 2H) 8.47 (d, J = 2.4 Hz, 1H) 7.88 (dd, J = 8.2 and 2.5 Hz, 1H) |
| | 7.70, (d, J = 2.4 Hz, 1H) 7.52 (d, J = 8.2 Hz, 1H) 7.36-7.29 (m, 2H) 7.14- |
| | 6.97 (m, 5H) 6.94 (dd, J = 8.8 and 2.4 Hz, 1H) 6.82-6.74 (m, 3H) 4.39 (d, |
| | J = 5.7 Hz, 2H) 3.92 (s, 2H). |
| 6:3 | 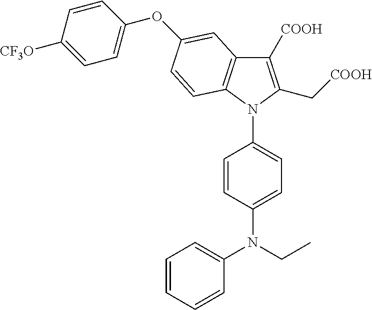 |
| | 2-Carboxymethyl-1-{4-[ethyl(phenyl)amino]phenyl}-5-(4-trifluoromethoxy- |
| | phenoxy)indole-3-carboxylic acid |
| | 12.43 (br s, 2H) 7.71 (dd, J = 2.4 and 0.3 Hz, 1H) 7.46-7.38 (m, 2H) 7.37- |
| | 7.30 (m, 2H) 7.27-7.20 (m, 4H) 7.19-7.14 (m, 1H) 7.10-7.02 (m, 3H) 7.00- |
| | 6.92 (m, 3H) 3.98 (s, 2H) 3.83 (q, J = 7 Hz, 2H) 1.19 (t, J = 7 Hz, 3H) |
| 6:4 | 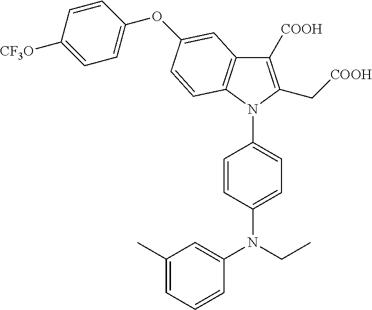 |
| | 2-Carboxymethyl-1-{4-[ethyl(m-tolyl)amino]phenyl}-5-(4-trifluoromethoxy- |
| | phenoxy)indole-3-carboxylic acid |
| | 12.47 (br s, 2H) 7.71 (d, J = 2.4 Hz, 1H) 7.35-7.27 (m, 3H) 7.23-7-17 (m, 2H) |
| | 7.10-6.99 (m, 6H) 6.96 (dd, J = 8.8 and 2.4 Hz, 1H) 6.94-6.89 (m, 2H) 3.94 (s, |
| | 2H) 3.81 (q, J = 7 Hz, 2H), 2.32 (s, 3H) 1.18 (t, J = 7 Hz, 3H) |
| 6:5 | 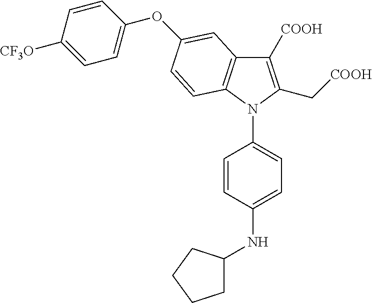 |
| | 2-Carboxymethyl-1-(4-cyclopentylaminophenyl)-5-(4-trifluoromethoxy- |
| | phenoxy)indole-3-carboxylic acid |
| | 12.39 (br s, 2H) 7.70 (d, J = 2.4 Hz, 1H) 7.36-7-30 (m, 2H) 7.10-7.00 (m, |
| | 5H) 6.95 (dd, J = 8.8 and 2.4 Hz, 1H) 6.75-6.68 (m, 2H) 6.11 (d, J = 6.2 |
| | Hz, 1H) 3.93 (s, 2H) 3.79-3.69 (m, 1H) 2.00-1.89 (m, 2H) 1.76-1.64 (m, |
| | 2H) 1.63-1.54 (m, 2H) 1.53-1.44 (m, 2H) |
| 6:6 | 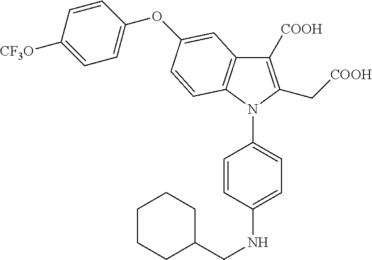 |
| | 2-Carboxymethyl-1-(4-cyclohexylmethylaminophenyl)-5-(4-trifluoro- |
| | methoxyphenoxy)indole-3-carboxylic acid |
| | 7.72 (d, J = 2.3 Hz, 1H) 7.36-7.29 (m, 2H) 7.09-6.96 (m, 5H) 6.90 (dd, J = 8.8 and |
| | 2.4 Hz, 1H) 6.74-6.67 (m, 2H) 6.10 (t, J = 5.3 Hz, 1H) 3.72 (s, 2H) 2.95-2.88 (m, |
| | 2H) 1.89-1.79 (m, 2H) 1.76-1.52 (m, 4H) 1.30-1.11 (m, 3H) 1.04-0.92 (m, 2H) |
| 6:7 | 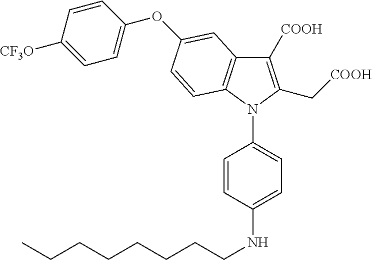 |
| | 2-Carboxymethyl-1-(4-octylaminophenyl)-5-(4-trifluoromethoxyphenoxy)- |
| | indole-3-carboxylic acid |
| | 12.38 (br s, 2H) 7.70 (d, J = 2.4 Hz, 1H) 7.36-7.30 (m, 2H) 7.10-6.99 (m, 5H) 6.95 |
| | (dd, J = 8.8 and 2.4 Hz, 1H) 6.74-6.68 (m, 2H) 6.08 (t, J = 5 Hz, 1H) 3.93 (s, 2H) |
| | 3.09-3.01 (m, 2H) 1.63-1.54 (m, 2H) 1.45-1.21 (m, 10H) 0.90-0.84 (m, 3H) |
| 6:8 | 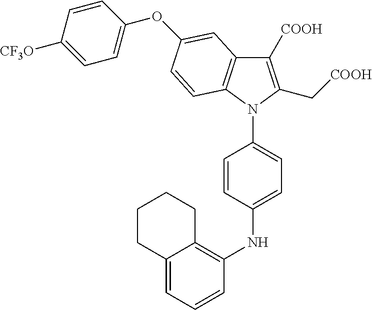 |
| | 2-Carboxymethyl-1-[4-(5,6,7,8-tetrahydro-1-naphthylamino)phenyl]-5- |
| | (4-trifluoromethoxyphenoxy)indole-3-carboxylic acid |
| | 7.72 (d, J = 2.4 Hz, 1H), 7.66 (s, 1H), 7.36-7.30 (m, 2H), 7.20-7.15 (m, 2H), 7.14- |
| | 6.97 (m, 7H), 6.93 (dd, J = 8.8 and 2.4 Hz, 1H), 6.82 (d, J = 7.2 Hz, 1H), 3.81 (s, |
| | 2H), 2.79-2.72 (m, 2H), 2.68-2.61 (m, 2H), 1.81-1.68 (m, 4H) |
| 6:9 | 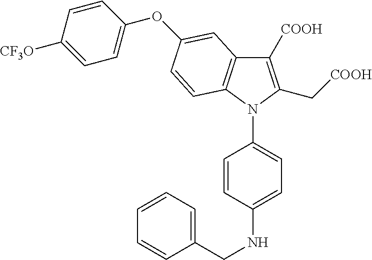 |
| | 1-(4-benzylaminophenyl)-2-carboxymethyl-5-(4-trifluoromethoxy- |
| | phenoxy)indole-3-carboxylic acid |
| | 12.38 (br s, 2H), 7.70 (d, J = 2.4 Hz, 1H), 7.45-7.40 (m, 2H), 7.39-7.30 (m, 4H), |
| | 7.29-7.23 (m, 1H), 7.10-6.97 (m, 5H), 6.94 (dd, J = 8.8 and 2.4 Hz, 1H), 6.79-6.70 |
| | (m, 3H), 4.33 (d, J = 5.4 Hz, 2H), 3.93 (s, 2H) |

### EXAMPLE 7

Title compounds of the Examples were tested in the biological in vitro assay Method A or B, described above, and were found to inhibit LTC₄ synthase. Thus, when the total concentration of title compounds in the assay was 10 μM, the following %-inhibition values where obtained.

| Ex. | % inh. | Method |
|---|---|---|
| 1:1 | 99 | B |
| 1:2 | 96 | B |
| 1:3 | 95 | B |
| 1:4 | 93 | B |
| 1:5 | 97 | B |
| 1:6 | 95 | B |
| 1:7 | 94 | B |
| 1:8 | 98 | B |
| 1:9 | 98 | B |
| 1:10 | 98 | A |
| 1:11 | 99 | B |
| 2:1 | 89 | A |
| 3:1 | 95 | B |
| 3:2 | 97 | B |
| 3:3 | 96 | B |
| 3:4 | 9 | B |
| 3:5 | 90 | B |
| 4:1 | 98 | B |
| 4:2 | 100 | B |
| 4:3 | 97 | B |
| 4:4 | 98 | B |
| 4:5 | 99 | B |
| | | B |
| | | B |
| 5:1 | 97 | B |
| 5:2 | 97 | B |
| 5:3 | 95 | B |
| 6:1 | 100 | B |
| 6:2 | 95 | B |
| 6:3 | 100 | B |
| 6:4 | 94 | B |
| 6:5 | 100 | B |
| 6:6 | 97 | B |
| 6:7 | 98 | B |
| 6:8 | 97 | B |
| 6:9 | 100 | B |

### EXAMPLE 8

Compounds of the Examples were tested in the biological in vivo assay described above using 1, 3 or 10 mg/kg of the exemplified compound. The following %-inhibition values where obtained.

Example 1:1, 1 mg/kg: 78%

Example 1:1, 3 mg/kg: 92%

Example 1:1, 10 mg/kg: 100%

Example 1:5, 10 mg/kg: 92%

Example 1:8, 10 mg/kg: 96%

Example 1:9, 3 mg/kg: 97%

Example 3:1, 3 mg/kg: 93%

Example 3:3, 10 mg/kg: 100%

Example 4:1, 10 mg/kg: 96%

Example 4:5, 3 mg/kg: 97%

Example 5:2, 3 mg/kg: 94%

1. A compound of formula I, 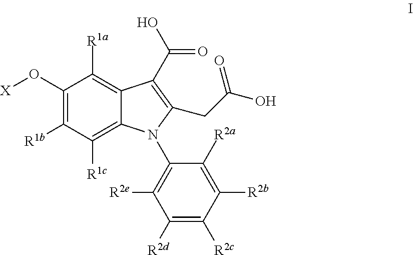
wherein:
R^{1a}, R^{1b} and R^{1c} independently represent hydrogen, halo, —CN or C₁₋₃ alkyl optionally substituted by one or more fluoro atoms;
any one of R^{2a}, R^{2b}, R^{2c}, R^{2d} and R^{2e} represents -L-Y, and the others independently represent hydrogen, halo, —CN or C₁₋₃ alkyl optionally substituted by one or more fluoro atoms;
X represents: 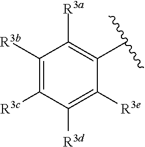
R^{3a}, R^{3b}, R^{3c}, R^{3d} and R^{3e} independently represent hydrogen, halo, —CN, R^{a}, —N(R^{b})R^{c}, —OR^{d} or —S(O)ₙR^{e};
L represents a direct bond, —N(R^{y})—, —O— or —S(O)ₘ—;
Y represents:
(i) G¹;
(ii) —CH₂-G²;
(iii) cyclopropyl optionally substituted by one or more substituents independently selected from G³;
(iv) ethyl, 1-propyl (i.e. n-propyl) or 2-propyl (i.e. isopropyl), all of which are substituted by one or more substituents independently selected from G⁴; or
(v) C₄₋₁₅ alkyl optionally substituted by one or more substituents independently selected from G⁵;
R^{a} and R^{e} independently represent C₁₋₆ alkyl optionally substituted by one or more fluoro atoms;
R^{b}, R^{c}, R^{d} and R^{y} independently represent hydrogen or C₁₋₆ alkyl optionally substituted by one or more fluoro atoms;
G¹ and G² independently represent an aryl group or a heteroaryl group, both of which groups are optionally substituted by one or more substituents independently selected from G⁶;
G³, G⁴ and G⁵ independently represent fluoro, —OR^{f}, aryl or heteroaryl, which latter two groups are optionally substituted by one or more substituents independently selected from G⁷;
G⁶ and G⁷ independently represent, on each occasion when used herein, halo, —CN, R^{g}, —N(R^{h})Rⁱ, —OR^{j} or —S(O)ₚR^{k};
R^{f}, R^{h}, Rⁱ and R^{j} independently represent, on each occasion when used herein, hydrogen or C₁₋₆ alkyl optionally substituted by one or more fluoro atoms;
R^{g} and R^{k} independently represent C₁₋₆ alkyl optionally substituted by one or more fluoro atoms;
n, m and p independently represent 0, 1 or 2,
or a pharmaceutically-acceptable salt thereof.

2. The compound according to claim 1, wherein R^{3c} represents —OR^{d}.

3. The compound according to claim 1, wherein R^{3a}, R^{3b}, R^{3d} and R^{3e} independently represent hydrogen.

4. The compound according to claim 1, wherein L represents —N(R^{y})— or —O—.

5. The compound according to claim 1, wherein R^{y} represents hydrogen or C₁₋₂ alkyl (e.g. ethyl).

6. The compound according to claim 1, wherein Y represents: (i) G¹; (ii) —CH₂-G²; or (iii) C₄₋₁₅ alkyl optionally substituted by one or more substituents selected from G⁵.

7. The compound according to claim 6, wherein G¹ and G² independently represent optionally substituted (i.e. by G⁶ or G⁷) phenyl, naphthyl, pyrrolyl, furanyl, thienyl, imidazolyl, oxazolyl, isoxazolyl, thiazolyl, pyrazolyl, pyridyl, indazolyl, indolyl, indolinyl, isoindolinyl, quinolinyl, 1,2,3,4-tetrahydroquinolinyl, isoquinolinyl, 1,2,3,4-tetrahydroisoquinolinyl, quinolizinyl, benzoxazolyl, benzofuranyl, isobenzofuranyl, chromanyl, benzothienyl, pyridazinyl, pyrimidinyl, pyrazinyl, indazolyl, benzimidazolyl, quinazolinyl, quinoxalinyl, 1,3-benzodioxolyl, tetrazolyl, benzothiazolyl, and/or benzodioxanyl.

8. The compound according to claim 7, wherein G¹ and G² independently represent phenyl, pyridyl, quinolinyl or naphthyl, all of which are optionally substituted by one or more G⁶ groups.

9. The compound according to claim 1, wherein G⁴ represents fluoro.

10. The compound according to claim 1, wherein G⁵ represents fluoro.

11. The compound according to claim 1, wherein G⁶ and G⁷ independently represent halo, —OR^{j}, R^{g} or —S(O)ₚR^{k}.

12. The compound according to claim 1, wherein R^{a} and R^{d} independently represent C₁₋₄ alkyl optionally substituted by one to three fluoro atoms; R^{b} and R^{c} independently represent hydrogen or C₁₋₄ alkyl optionally substituted by one to three fluoro atoms; R^{j} represents hydrogen or C₁₋₃ alkyl optionally substituted by one to three fluoro atoms; and/or R^{g} and R^{k} independently represent C₁₋₃ alkyl optionally substituted by one to three fluoro atoms.

13. The compound according to claim or a pharmaceutically-acceptable salt thereof, for use as a pharmaceutical.

14. A pharmaceutical formulation including a compound as defined in claim 1, or a pharmaceutically-acceptable salt thereof, in admixture with a pharmaceutically acceptable adjuvant, diluent or carrier.

15. A method of treatment of a disease in which inhibition of the synthesis of leukotriene C₄ is desired and/or required by administration of a compound according to claim 1 or a pharmaceutical salt thereof.

16. A method of manufacture of a medicament for the treatment of a disease in which inhibition of the synthesis of leukotriene C₄ is desired and/or required comprising combining a compound according to claim 1, or a pharmaceutically acceptable salt thereof with a pharmaceutically acceptable carrier.

17. The method according to claim 15 or 16, wherein the disease is a respiratory disease, inflammation and/or has an inflammatory component.

18. A compound or use as claimed in claim 17 wherein the disease is an allergic disorder, asthma, childhood wheezing, a chronic obstructive pulmonary disease, bronchopulmonary dysplasia, cystic fibrosis, an interstitial lung disease, an ear nose and throat disease, an eye disease, a skin diseases, a rheumatic disease, vasculitis, a cardiovascular disease, a gastrointestinal disease, a urologic disease, a disease of the central nervous system, an endocrine disease, urticaria, anaphylaxis, angioedema, oedema in Kwashiorkor, dysmenorrhoea, a burn-induced oxidative injury, multiple trauma, pain, toxic oil syndrome, endotoxin chock, sepsis, a bacterial infection, a fungal infection, a viral infection, sickle cell anaemia, hypereosinofilic syndrome, or a malignancy

19. A compound or use as claimed in claim 18, wherein the disease is an allergic disorder, asthma, rhinitis, conjunctivitis, COPD, cystic fibrosis, dermatitis, urticaria, an eosinophilic gastrointestinal disease, an inflammatory bowel disease, rheumatoid arthritis, osteoarthritis or pain

20. A method of treatment of a disease in which inhibition of the synthesis of leukotriene C₄ is desired and/or required, which method comprises administration of a therapeutically effective amount of a compound of formula I as defined in claim 1, or a pharmaceutically-acceptable salt thereof, to a patient suffering from, or susceptible to, such a condition

21. A combination product comprising:
(A) a compound of formula I as defined in claim 1, or a pharmaceutically-acceptable salt thereof; and
(B) another therapeutic agent that is useful in the treatment of a respiratory disorder and/or inflammation,
wherein each of components (A) and (B) is formulated in admixture with a pharmaceutically-acceptable adjuvant, diluent or carrier.

22. The combination product according to claim 21 which comprises a pharmaceutical formulation including a compound of formula I as defined in claim 1, or a pharmaceutically-acceptable salt thereof, another therapeutic agent that is useful in the treatment of a respiratory disorder and/or inflammation, and a pharmaceutically-acceptable adjuvant, diluent or carrier.

23. The combination product according to claim 21 which comprises a kit of parts comprising components:
(a) a pharmaceutical formulation including a compound of formula I as defined in claim 1, or a pharmaceutically-acceptable salt thereof, in admixture with a pharmaceutically-acceptable adjuvant, diluent or carrier; and
(b) a pharmaceutical formulation including another therapeutic agent that is useful in the treatment of a respiratory disorder and/or inflammation in admixture with a pharmaceutically-acceptable adjuvant, diluent or carrier,
which components (a) and (b) are each provided in a form that is suitable for administration in conjunction with the other

24. A process for the preparation of a compound of formula I as defined in claim 1, which process comprises:
(i) reaction of a compound of formula II, 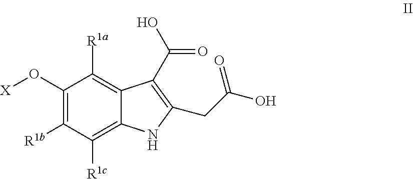
wherein R^{1a}, R^{1b}, R^{1c} and X are as defined in claim 1, with a compound of formula III, 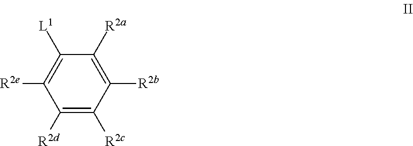
wherein L¹ represents a suitable leaving group;
(ii) reaction of a compound of formula IV, 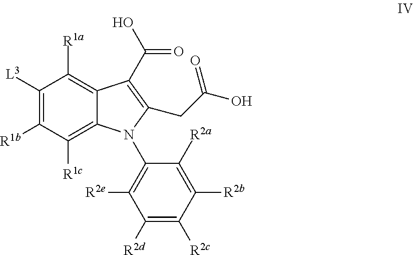
wherein L³ represents a suitable leaving group, and R^{1a} to R^{1c} and R^{2a} to R^{2e} are as defined in claim 1 with a compound of formula V, 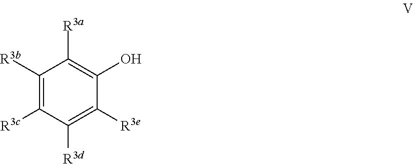
wherein R^{3a} to R^{3e} are as defined in claim 1;
(iii) reaction of a compound of formula VI, 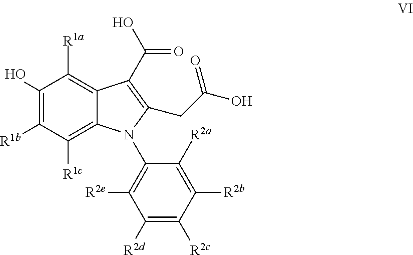
wherein R^{1a} to R^{1c} and R^{2a} to R^{2e} are as defined in claim 1 with a compound of formula VII, 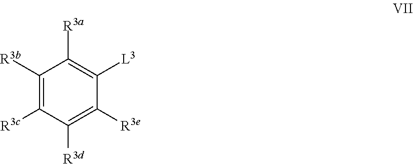
wherein L³ is as defined above, and R^{3a} to R^{3e} are as defined in claim 1;
(iv) reaction of a protected derivative of a compound of formula VIII, 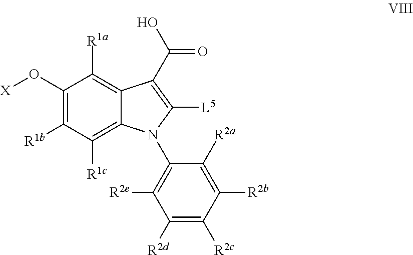
wherein L⁵ represents an appropriate alkali metal group, a —Mg-halide, a zinc-based group or a suitable leaving group, the —COOH is protected, and R^{1a} to R^{1c}, R^{2a} to R^{2e} and X are as defined in claim 1, with a compound of formula IX, 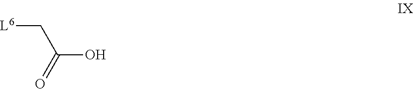
wherein the —COOH is protected, and L⁶ represents a suitable leaving group;
(v) reaction of a protected derivative of a compound of formula X, 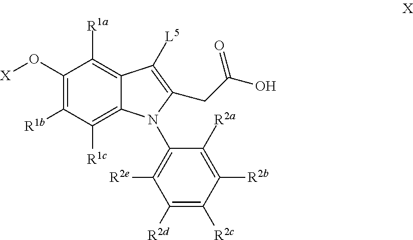
wherein the —COOH is protected, and L⁵, R^{1a} tom R^{1c}, R^{2a} to R^{2e} and X are as defined in claim 1,
with a protected derivative of a compound of formula XI,
L⁶-C(O)OH XI
wherein the —COOH is protected, and L⁶ is as defined above;
(vi) reaction of a compound of formula X as defined above in which L⁵ represents either:
(I) an alkali metal; or
(II) —Mg-halide,
with carbon dioxide, followed by acidification under standard conditions known to those skilled in the art, for example, in the presence of aqueous hydrochloric acid;
(vii) for compounds of formula I, and protected derivatives thereof, reaction of a corresponding compound of formula X as defined above in which L⁵ is a suitable leaving group with CO (or a reagent that is a suitable source of CO), in the presence of a compound of formula XII,
R⁴—OH XII
wherein R⁴ represents hydrogen or a suitable protecting group;
(viii) for compounds of formula I in which L represents —O—, —N(R^{y})— or —S—, reaction of a compound corresponding to a compound of formula I, but in which Y represents hydrogen, with a compound of formula XIII,
L⁷-Y XIII
wherein L⁷ represents a suitable leaving group;
(ix) for compounds of formula I in which L represents —O—, —N(R^{y})— or —S—, reaction of a compound corresponding to a compound of formula I, but in which -L-Y represents a suitable leaving group, with a compound of formula XIV,
L^{x1}-Y XIV
wherein L^{x1} represents —OH, —N(R^{y})H or —SH, and R^{y} and Y are as defined in claim 1.

25. A process for the preparation of a pharmaceutical formulation as defined in claim 14, which process comprises bringing into association a compound of formula I, as defined in claim 1, or a pharmaceutically acceptable salt thereof with a pharmaceutically-acceptable adjuvant, diluent or carrier.

26. A process for the preparation of a combination product according to claim 21, which process comprises bringing into association a compound of formula I, as defined in claim 1, or a pharmaceutically acceptable salt thereof with the other therapeutic agent that is useful in the treatment of a respiratory disorder and/or inflammation, and at least one pharmaceutically-acceptable adjuvant, diluent or carrier.

27. A process for the preparation of a combination according to claim 22, which process comprises bringing into association a compound of formula I, as defined in claim 1, or a pharmaceutically acceptable salt thereof with the other therapeutic agent that is useful in the treatment of a respiratory disorder and/or inflammation, and at least one pharmaceutically-acceptable adjuvant, diluent or carrier.

28. A process for the preparation of a combination according to claim 23, which process comprises bringing into association a compound of formula I, as defined in claim 1, or a pharmaceutically acceptable salt thereof with the other therapeutic agent that is useful in the treatment of a respiratory disorder and/or inflammation, and at least one pharmaceutically-acceptable adjuvant, diluent or carrier.
